(19) United States
(12) Patent Application Publication
(10) Pub. No.: US 2009/0191282 A1
(43) Pub. Date: Jul. 30, 2009

(54) CONTROLLED RELEASE COMPOSITION CONTAINING A STRONTIUM SALT

(75) Inventors: Christian Hansen, Vedbaek (DK); Henrik Nilsson, Copenhagen (DK); Jens E.T. Andersen, Vedbaek (DK); Stephan Christgau, Gentofte (DK)

Correspondence Address: JONES DAY, 222 EAST 41ST ST, NEW YORK, NY, 10017, US

(73) Assignee: Osteologix A/S

(21) Appl. No.: 12/341,439

(22) Filed: Dec. 22, 2008

Related U.S. Application Data
(63) Continuation of application No. 10/556,150, filed on Jun. 8, 2006, now abandoned.
(60) Provisional application No. 60/528,409, filed on Dec. 9, 2003.

(30) Foreign Application Priority Data
May 7, 2003 (DK) PA 2003 00691
Jul. 8, 2003 (DK) PA 2003 01043
Dec. 9, 2003 (DK) PA 2003 01821

(51) Int. Cl. A61K 33/14; A61K 31/19; A61K 31/195; A61K 31/194; A61P 19/00; A61K 33/06; A61K 31/59

(52) U.S. Cl. 424/677; 514/557; 514/561; 514/574; 424/682; 514/171

(57) ABSTRACT

A controlled release pharmaceutical composition comprising a strontium salt. The invention also relates to the use of a strontium salt for treating a male suffering from diseases and conditions affecting metabolism and/or structural integrity of cartilage and/or bone. The invention also relates to the use of a strontium-containing compound for preventing a cartilage and/or bone condition in a subject, and for the treatment and/or prophylaxis of secondary osteoporosis.

### CROSS REFERENCE TO RELATED APPLICATIONS

This application is a continuation of application Ser. No. 10/556,150, filed Jun. 8, 2006, which is the National Stage of International Application No. PCT/DK04/00326, filed May 6, 2004, which claims the benefit of priority of Provisional Application No. 60/528,409, filed Dec. 9, 2003; and which also claims the benefit of Denmark Application No. PA 200300691, filed May 7, 2003; Denmark Application No. PA 200301043, filed Jul. 8, 2003; and Denmark Application No. PA 200301821, filed Dec. 9, 2003. Application Ser. No. 10/556,150, filed Jun. 8, 2006 is hereby incorporated by reference in its entirety.

### FIELD OF THE INVENTION

The present invention relates to a controlled release pharmaceutical composition comprising a strontium salt. The invention also relates to the use of a strontium salt for treating a male suffering from diseases and conditions affecting metabolism and/or structural integrity of cartilage and/or bone. The invention also relates to the use of a strontium-containing compound for preventing a cartilage and/or bone condition in a subject, and for the treatment and/or prophylaxis of secondary osteoporosis.

### BACKGROUND OF THE INVENTION

Osteoporosis is the most common form of metabolic bone disease in humans. It is a condition, which affects a very large number of people all over the world, and as the number of elderly people is set to rise dramatically in the coming decades in most countries, the prevalence and impact of osteoporosis will also increase. The disease is characterized pathologically by an absolute decrease in the amount of bone mass and the structural quality of bone, and clinically by increased susceptibility to fractures. In fact, osteoporosis is the most significant underlying cause of skeletal fractures in late middle aged and elderly women.

In general, there are two types of osteoporosis: primary and secondary. Secondary osteoporosis is the result of an identifiable disease process, treatments or therapeutic agents. However, approximately 90% of all osteoporosis cases are idiopathic primary osteoporosis. Such primary osteoporosis includes postmenopausal osteoporosis, age-associated osteoporosis (affecting a majority of individuals over the age of 70 to 80), and idiopathic osteoporosis affecting middle-aged and younger men and women.

The mechanism of bone loss in osteoporosis is believed to involve an imbalance in the process of bone remodeling. Bone remodeling occurs throughout life, renewing the skeleton and maintaining the strength of bone. This remodeling is mediated by specialized cells of the bone tissue, called “osteoclasts” and “osteoblasts”. Osteoclasts (bone dissolving or resorbing cells) are responsible for the resorption of a portion of bone within the bone matrix, during the resorption process. After resorption, the osteoclasts are followed by the appearance of osteoblasts (bone forming cells), which then refill the resorbed portion with new bone.

The formation of the two cell types as well as their activity in bone is usually tightly coupled and well regulated in order to maintain the skeletal balance and structural integrity of the bones. However, in people with osteoporosis an imbalance in this remodeling process develops, resulting in loss of bone at a rate faster than the accretion of bone.

The single most important risk factor for osteoporosis is oestrogen deficiency occurring naturally at the menopause. The decline in endogenous oestrogen production leads to an elevated metabolic activity in the bone tissue where the increase in osteoclast mediated bone resorption surpass the more modest increase in bone formation resulting in a net loss of bone. The actual number of people affected will grow at a rate greater than simple population growth rates, because the aging of the population is disproportionately increasing the older segment of the population, while the age for the onset of menopause has remained constant. In the last decades there has also been a substantial advance in the ability to predict and monitor osteoporosis, as methods for measurement of bone mineral density (BMD) has improved and new specific biochemical markers of bone resorption and formation has been developed and made available for routine clinical use. New pharmaceutical agents for treatment and/or prevention of osteoporosis have also been developed. The majority of these treatments are either based on substituting the lost endogenous estrogen either in the form of hormone replacement therapy (HRT) or selective estrogen receptor modulators (SERM), or they belong to the class of compounds called bisphosphonates. SERM's and especially HRT can only be administered to female subjects as administration of estrogen and estrogen like substances to a male will be associated with unwanted hormonal effect. Furthermore even in women the use of SERMs and especially HRT is associated with significant side effects, such as increased risk of cancer and cardiovascular disease, whereas bisphosphonates in addition to a potent antiresorptive effect also decreases bone formation to a similar extent, implying that they loose their therapeutic effect after few years of treatment. Thus, there is a need for agents, which are effective in the treatment and/or prophylaxis of osteoporosis.

### SUMMARY OF THE INVENTION

In one aspect, the invention relates to a controlled release pharmaceutical composition for oral use comprising a strontium salt, wherein the amount of strontium salt is adjusted so that the composition is suitable for administration once daily.

In one embodiment of the composition, the water-solubility of the strontium salt is at the most about 200 g/l such as, at the most about 150 g/l, at the most about 100 g/l, at the most about 75 g/l, at the most about 50 g/l, at the most about 25 g/l, at the most about 10 g/l, at the most about 5 g/l, at the most about 2.5 g/l, or at the most about 1 g/l at room temperature (20-25° C.). In certain embodiments of the composition, the water solubility of the strontium salt is at least 0.1 g/l such as, in a range of from about 0.1 g/l to about 10 g/l, from 15 about 0.2 g/l to about 5 g/l at room temperature (20-25° C.). In specific embodiments of the composition, the water solubility of the strontium salt is at least 1 g/l, such as, at least 5 g/l, at least 10 g/l, at least 20 g/l, at least 30 g/l, at least 40 g/l, at least 50 g/l, at least 60 g/l, at least 70 g/l, at least 80 g/l, at least 90 g/l or at least 100 g/l at room temperature of (20-25° C.).

In specific embodiments, the composition can be administered once daily at bed-time.

In some embodiments of the composition, the composition comprises at least 0.01 g of strontium (calculated as ionic strontium), such as, at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as, from about 0.1 to about 2 g, from about 0.1 to about 1 g, from about 0.15 to about 0.5 g, from about 0.3 to about 2 g or from about 0.3 to about 1 g. In certain embodiments, the composition comprises at least about 0.5 g of strontium (calculated as ionic strontium) such as, at least about 0.6 g, at least about 0.7 g least 0.8 g, at least 0.9 g, at least 1.0 g, at least 1.1 g, at least 1.2 g, at least 1.3 g, at least 1.4 g, at least 1.5 g, at least 1.6 g, at least 1.7 g, at least 1.8 g, at least 1.9 g or at least 2.0 g.

In specific embodiments of the composition, the Sr salt is released from the composition in such a manner that the amplitude (difference between peak and nadir) of the plasma concentration relative to the peak level should be less than about 40% such as, less than about 35%, less than about 30%, less than about 25%, less than about 20%, less than about 15% or less than about 10% after administration of the composition to a subject once daily for at least 30 days such as, at least 21 days, at least 14 days, at least 7 days such as 7 days.

In some embodiments, the composition when tested in an in vitro dissolution test-releases strontium ion from the strontium salt containing composition in the following manner:

within the first 30 min of the test at the most about 10% w/w of the Sr ion is released;

within the first 4 hours of the test at the most about 70% w/w of the Sr ion is released; and

within the first 14 hours of the test about 70% w/w or more of the Sr ion is released.

In some embodiments of the composition, the Sr salt is contained in a matrix that governs the release.

In some embodiments of the composition, the composition is coated with a controlled release coating that governs the release of the Sr salt.

In certain embodiments of the composition, the strontium salt is selected from the group consisting of strontium salts of an organic or an inorganic acid. The anion of the strontium salts acid can be, for instance, derived from a monoprotic, a diprotic or a triprotic acid.

In embodiments of the pharmaceutical composition wherein the strontium salt is an inorganic acid, the acid can be, for example, hydrofluoric acid, hydrochloric acid, hydrobromic acid, hydroiodic acid, nitric acid, nitrous acid, phosphoric acid, phosphinic acid, phosphonic acid, sulfonic acid, sulfuric acid, sulfurous acid, disulfuric acid or boric acid.

In embodiments of the pharmaceutical composition wherein the strontium salt is an inorganic acid, the acid can be, for example, acetic acid, C₂H₅COOH, C₃H₇COOH, C₄H₉COOH, (COOH)₂, CH₂(COOH)₂, C₂H₄(COOH)₂, C₃H₆(COOH)₂, C₄H₈(COOH)₂, C₅H₁₀(COOH)₂, fumaric acid, maleic acid, malonic acid, lactic acid, citric acid, tartaric acid, oxalic acid, ascorbic acid, benzoic acid, salicylic acid, phthalic acid, carbonic acid, formic acid, acid, L- and D-glutamic acid, L- and D-aspartic acid, glucosamine sulphate, L-threonate, trifluoroacetic acid and ranelic acid.

In specific embodiments of the pharmaceutical composition, the acid is a non-chelator of strontium.

In specific embodiments of the pharmaceutical composition, the salt is in hydrate, anhydrous, solvate, polymorphous, amorphous, crystalline, microcrystalline or polymeric form.

In certain embodiments of the pharmaceutical composition, the salt is selected from the group comprising strontium citrate, strontium succinate, strontium fumarate, strontium ascorbate, strontium tartrate, strontium glutarate, strontium malonate, strontium methanesulfonate, strontium benzenesulfonate, strontium glucosamine sulphate, strontium L-threonate, and mixtures thereof.

In another aspect, the invention relates to a method for treatment and/or prophylaxis of a cartilage and/or bone disease and/or conditions resulting in a dysregulation of cartilage and/or bone metabolism in a mammal, such as a human female or male adult, adolescent or child, such as osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues and for weight gain, the method comprising administering a single daily dose of a Sr salt comprising at least 0.5 g of strontium (calculated as strontium ion), such as, at least 0.6 g, at least about 0.7 g at least 0.8 g, at least 0.9 g, at least 1.0 g, at least 1.1 g, at least 1.2 g, at least 1.3 g, at least 1.4 g, at least 1.5 g, at least 1.6 g, at least 1.7 g, at least 1.8 g, at least 1.9 g or at least 2.0 g.

In still another aspect, the invention relates to a method for treatment and/or prophylaxis of a cartilage and/or bone disease and/or conditions resulting in a dysregulation of cartilage and/or bone metabolism in a male mammal, such as a human male adult, adolescent or child, such as osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues and for weight gain, the method comprising administering a Sr salt in an amount and frequency that gives a daily dose of from about 0.25 g to about 1.5 g free Sr²⁺, such as, from about 0.30 g to about 1.5 g, from about 0.40 g to about 1.40 g, from about 0.50 g to about 1.30 g, from about 0.60 g to about 1.20 g, from about 0.70 g to about 1.10 g or from about 0.80 g to about 1.00 g.

In one embodiment of this method, the Sr salt is administered orally.

In some embodiments of this method, the Sr salt is contained in a pharmaceutical composition, which can be, for example, of the type described above.

Another aspect of the invention relates to a method for preventing in a subject a cartilage and/or bone disease and/or conditions resulting in a dysregulation of cartilage and/or bone metabolism in a mammal, such as a human male or female adult, adolescent or child, such as, osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, and for the maintenance or increase of energy level, for building up or strengthening muscle tissues and for weight gain, the method comprising administering a Sr salt.

In a specific embodiment of the method, the subject is a female having a bone mineral density, BMD, of more than 1 standard deviation (SD) below the young adult female mean.

In another specific embodiment of the method, the subject is a female having a BMD below the adult female mean for women of the same age.

In a specific embodiment of the method, the subject is a male having a BMD of more than 1 SD below the young adult male mean.

In another specific embodiment of the method, the subject is a male having a BMD below the adult male mean for men of the same age.

In another specific embodiment of the method, the subject is a female having a level of a specific biomarker of bone resorption, of more than 1 SD above the young adult female mean.

In another specific embodiment of the method, the subject is a female having a level of a specific biomarker of bone resorption above the adult female mean for women of the same age.

In another specific embodiment of the method, the subject is a male having a level of a specific biomarker of bone resorption, of more than 1 SD above the young adult male mean.

In another specific embodiment of the method, the subject is a male having a level of a specific biomarker of bone resorption above the adult mean for men of the same age.

In another specific embodiment of the method, the subject is a 20 year or older such as 25 years or older, 30 years or older, 35 years or older, 40 years or older, 45 years or older, or 50 years or older female.

In a specific embodiment of the method, the subject is a female that is about the same age as her age of onset of menopause.

In another specific embodiment of the method, the subject is a female who is about 6 months or more beyond the onset of menopause.

In another specific embodiment of the method, the subject is a 20 year or older such as, 25 years or older, 30 years or older, 35 years or older, 40 years or older, 45 years or older, 50 years or older, 55 years or older, 60 years or older, 65 years or older, or 70 years or older male.

In some embodiments of the method, the daily dose of strontium administered is at least 0.01 g of strontium, such as, at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as, from about 0.1 to about 2 g, from about 0.1 to about 1 g, from about 0.15 to about 0.5 g, from about 0.3 to about 2 g or from about 0.3 to about 1 g.

In some embodiments, the method further comprises administering an amount of calcium to a subject in need thereof. In some embodiments, the daily dose of calcium is at least about 0.01 g, such as, at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as from about 0.1 to about 2 g, from about 0.5 to about 2 g, from about 0.5 g to about 1 g, or from about 1 to about 1.5 g. The calcium can be administered, for example, at least 0.5 h, such as at least 1 h, at least 2 h, at least 3 h, at least 4 h, at least 5 h, at least 6 h, at least 7 h, at least 8 h, at least 9 h, at least 10 h, at least 11 h or at least 12 h after the administration of the strontium component. In certain embodiments, the calcium is administered at least 0.5 h, such as at least 1 h, at least 2 h, at least 3 h, at least 4 h, at least 5 h, at least 6 h, at least 7 h, at least 8 h, at least 9 h, at least 10 h, at least 11 h or at least 12 h before the administration of the strontium component.

In some embodiments, the method further comprises administering an amount of vitamin D to a subject in need thereof.

In certain embodiments, the strontium and the vitamin D components are administered simultaneously.

In some embodiments, the vitamin is vitamin D₃ and the daily dose is at least about 1 μg, such as, at least about 1.25 μg at least about 1.50 μg, at least about 2 μg, at least about 31 μg, at least about 4 μg, at least about 5 μg, at least about 10 μg, at least about 15 μg, at least about 20 μg, at least about 25 μg, at least about 30 μg, at least about 40 μg or at least about 50 μg or from about 1 μg to about 50 μg such as, from about 1.50 μg to about 40 μg, from about 2 μg to about 30 μg, from about 3 μg to about 30 μg, from about 41 μg to about 30 μg, from about 5 μg to about 30 μg, from about 10 μg to about 30 μg, from about 10 μg to about 20 μg or from about 15 μg to about 25 μg. In a specific embodiment, the daily dose of vitamin D₃ is from about 5 μg to about 30 μg, such as, from about 10 μg to about 20 μg.

In other embodiments, the vitamin D is vitamin D₂, and the daily dose of vitamin D₂ is at least 1 μg, such as, at least about 1.50, μg, at least about 2 μg, at least about 31 ug, at least about 4 μg, at least about 5 μg, at least about 10 μg, at least about 15 μg, at least about 20 μg, at least about 25 μg, at least about 30 μg, at least about 40 μg, at least about 50 μg, at least about 60 μg, at least about 70 μg, at least about 80 μg, at least about 90 μg, at least about 100 μg, at least about 110 μg, at least about 120 μg or at least about 125 μg or from about 1 μg to about 125 μg such as, from about 1.50 to about 120 μg, from about 2 μg to about 110 μg, from about 3 μg to about 100 μg, from about 4 μg to about 90 μg, from about 5 μg to about 80 μg, from about 5 μg to about 125 μg, from about 10 μg to about 70 μg, from about 10 μg to about 60 μg, from about 10 μg to about 50 μg, from about 10 μg to about 40 μg, from about 10 μg to about 30 μg, from about 10 μg to about 20 μg, or from about 15 μg to about 25 μg. In a specific embodiment, the daily dose of vitamin D₂ is from about 5 μg to about 125 μg, such as, from about 10 μg to about 20 μg.

Another aspect of the invention relates to a method for treating and/or preventing secondary osteoporosis in a subject, the method comprising administering an effective amount of a Sr salt to the subject. The secondary osteoporosis can be induced by, for instance, endocrine diseases, metabolic causes, nutritional conditions, drug substances and/or disorders of the collagen metabolism.

In still another aspect, the invention relates to a method for preventing drug induced secondary osteoporosis in a subject, the method comprising administering to the subject a prophylactic amount of a Sr salt before, during or after treatment of the subject with the drug substance that induces secondary osteoporosis. In certain embodiments of the invention, the administration takes place substantially simultaneously with administration of the drug substance that induces osteoporosis. In a specific embodiment of the invention, the Sr salt and the drug substance that induces osteoporosis are contained in the same pharmaceutical composition.

Another aspect of the invention relates to a pharmaceutical composition comprising a Sr salt and a drug substance that induces osteoporosis together with a pharmaceutically acceptable excipient.

### BRIEF DESCRIPTION OF FIGURES

FIG. 1 shows an X-ray diffractogram of crystals of strontium glutamate hexahydrate prepared by the method as described in Example 7.

FIG. 2 shows an X-ray diffractogram of crystals of strontium malonate prepared by the method as described in Example 7.

FIG. 3 graphically depicts the results of the optimization experiments for strontium glutamate synthesis outlined in Table 6. The influence on the yield of the synthesis of strontium glutamate was investigated by varying four parameters. (Yields above 100% indicate incomplete drying.)

FIG. 4 shows a plot of serum strontium concentrations measured in rats given a single dose of strontium as indicated in the upper part of each panel. The data points represent mean and standard deviation for each measuring point. Pre-dose represent corresponding samples taken from animals treated with vehicle alone.

### DESCRIPTION OF THE INVENTION

In a first aspect of the invention, it concerns a controlled release pharmaceutical composition for oral use comprising a strontium (Sr) salt. The composition is intended for administration once daily. In a specific aspect of the invention, the strontium salt is characterized by having a water solubility of at the most about 200 g/l at room temperature and in a specific aspect, the strontium salt has a relatively low water solubility under physiological conditions (i.e., a solubility below 1 g/l at 40° C.).

In a second aspect of the invention it relates to a pharmaceutical composition containing a strontium salt, wherein the composition is adapted to release the Sr salt in such a manner that the amplitude (difference between peak and nadir) of the plasma concentration relative to the peak level should be less than about 40% such as, e.g., less than about 35%, less than about 30%, less than about 25%, less than about 20%, less than about 15% or less than about 10% after administration of the composition to a subject once daily for a time period of 7 days or more. In a preferred aspect, the time period is 7 days.

In one embodiment of the invention the plasma concentration may fluctuate from about 16.2+/−3 mg/l to 20.0+/−2.3 mg/l Sr after administration of a pharmaceutical composition comprising a daily dose of approximately 650 mg ionic strontium.

In the present context, a controlled release pharmaceutical composition denotes a composition that has been designed to release the active substance (the strontium ion in solution) in a manner that is modified compared to the release from plain tablet. A person skilled in the art will know how to judge whether the release is controlled. Many other terms are normally employed to denote a controlled release such as, e.g., modified release, sustained release, delayed release, pulsatile release, prolonged release etc. All these terms are included in the term “controlled release” as used herein.

It is contemplated that therapy with Sr salts would be significantly improved by reducing the frequency of administration. Firstly, it is possible to reduce or minimize unwanted side effects and moreover, it is possible to achieve a plasma level that is constant or substantially constant during a prolonged period of time, i.e., leading to a reduction in the amplitude between the peak and the nadir values of the plasma concentration. Accordingly, the patient will potentially have a more efficient treatment with a sustained treatment effect (e.g., continuous anti-osteoporotic effect) during the treatment period.

A suitable in vitro method for determining whether a specific composition has suitable properties with respect to controlled release of the Sr salt is an in vitro dissolution test as described in Ph. Eur. Thus, a controlled release composition according to the invention—when tested in an in vitro dissolution test—releases strontium ion from the Sr salt containing pharmaceutical composition in the following manner:

within the first 30 min of the test at the most about 10% w/w of the Sr ion is released within the first 4 hours of the test at the most about 70% w/w of the Sr ion is released within the first 14 hours of the test about 70% w/w or more of the Sr ion is released.

The controlled release composition may be a composition, wherein the Sr salt is contained in a matrix that governs the release.

The composition may also be coated with a controlled release coating governing the release of the Sr containing compound.

Some of the known strontium salts (e.g., strontium chloride, strontium hydroxide) have a very high water-solubility (i.e., above 200 g/l in water at room temperature 20-25° C.). Irrespective of their water-solubility such strontium salts maybe incorporated into a controlled release composition for once daily administration. However, in a specific embodiment of the invention the water-solubility of the strontium salt is at the most about 200 g/l such as, e.g., at the most about 150 g/l, at the most about 100 g/l, at the most about 75 g/l, at the most about 50 g/l, at the most about 25 g/l, at the most about 10 g/l, at the most about 5 g/l, at the most about 2.5 g/l, or at the most about 1 g/l at room temperature (20-25° C.).

In those cases where e.g., a strontium salt having a water-solubility of at the most about 1 g/l (e.g., strontium citrate, strontium carbonate, strontium oxalate, strontium sulphate or strontium hydrogen phosphate), the present inventors have shown that it is possible to delay the appearance of the peak concentration, i.e., the active substance itself contributes to the controlled release and not only the design of the pharmaceutical composition.

Furthermore, the invention relates to a composition, wherein the amount of the Sr salt is adjusted so that the composition is suitable for administration once or twice daily.

As mentioned above, the composition may be suitable for administration once daily, e.g., at bedtime. It is known that bone resorption is higher during the night than during daytime, why the administration of an amount of Sr at bedtime could prove to be favorable compared to administration of a similar amount of Sr in the morning. As shown in the examples herein, a number of strontium salts, i.e., those that has a water solubility of less then 200 g/l (as mentioned above) have a delayed appearance of peak concentration of the ionic strontium compared, e.g., to that of the highly water soluble strontium chloride. Accordingly, such salts are contemplated to be suitable for use in designing a controlled release pharmaceutical composition containing a strontium salt.

The daily dose of strontium ion may be at least about 0.01 g, such as, e.g., at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as, e.g., from about 0.1 to about 2 g, from about 0.1 to about 1 g, from about 0.15 to about 0.5 g, from about 0.3 to about 2 g or from about 0.3 to about 1 g.

In a specific embodiment the invention also relates to a composition, comprising at least 0.5 g of Sr, defined as free ionic strontium, such as, e.g., at least 0.6 g, at least 0.7 g, at least 0.8 g, at least 0.9 g, at least 1.0 g, at least 1.1 g, at least 1.2 g, at least 1.3 g, at least 1.4 g, at least 1.5 g, at least 1.6 g, at least 1.7 g, at least 1.8 g, at least 1.9 g or at least 2.0 g daily.

In another embodiment, the invention also relates to a pharmaceutical composition, where the amount of strontium salt as well as pharmaceutical excipients have been adjusted so the composition is suitable for administration of the strontium compound with a frequency less than once daily, i.e., 3 times a week, 2 times a week or most preferred once a week.

Furthermore, the invention relates to a method for the treatment and/or prophylaxis of a cartilage and/or bone disease and/or conditions resulting in a dysregulation of cartilage and/or bone metabolism in a mammal, such as e.g., a human female or male adult, adolescent or child, such as, e.g., osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues and for weight gain, the method comprising administering a single daily dose of a Sr salt comprising at least 0.7 g Sr, such as, e.g., at least 0.8 g, at least 0.9 g, at least 1.0 g, at least 1.1 g, at least 1.2 g, at least 1.3 g, at least 1.4. g, at least 1.5 g, at least 1.6 g, at least 1.7 g, at least 1.8 g, at least 1.9 g or at least 2.0 g.

### Strontium

Previous studies have shown that various strontium compounds modulate bone loss in osteoporosis when present at levels higher than those required for normal cell physiology. The effect is believed to be due to a stimulatory effect of strontium on pre-osteoblastic cell replication, and a direct or matrix-mediated inhibition of osteoclast activity by strontium (Reginster, J Y, Curr pharm Des 2002:8 (21):1907-16). In other words, strontium both works as an anti-resorptive and an anabolic agent. Various salts of strontium are known from the prior art, such as, e.g., strontium ranelate (distrontium salt of 2-[N,N-di(carboxymethyl)amino]-3-cyano-4-carboxymethylthiophene-5-carboxylic acid) described in EP-B 0 415 850. The ranelate part of the strontium compound, derived from ranelic acid, is unlikely to have any therapeutic effect towards cartilage or bone conditions per se.

In principle any strontium-containing compound can be incorporated in a controlled release pharmaceutical composition according to the invention, provided that it is safe.

The following strontium salts of organic or inorganic acids may be in a composition as described above. The salts may be in hydrate, anhydrous, solvate, polymorphous, amorphous, crystalline, microcrystalline or polymeric form. In one embodiment of the invention only non-radioactive isotopes of Sr are used.

The inorganic acid for making strontium salts may be selected from the group consisting of boric acid, bromous acid, carbonic acid, chloric acid, diphosphoric acid, disulfuric acid, dithionic acid, dithionous acid, fulminic acid, hydrazoic acid, hydrobromic acid, hydrochloric acid, hydrofluoric acid, hydroiodic acid, hydrogen sulfide, hypophosphoric acid, hypophosphorous acid, iodic acid, iodous acid, metaboric acid, metaphosphoric acid, metaphosphorous acid, metasilicic acid, nitric acid, nitrous acid, orthophosphoric acid, orthophosphorous acid, orthosilicic acid, phosphoric acid, phosphinic acid, phosphonic acid, phosphorous acid, pyrophosphorous acid, selenic acid, sulfonic acid, sulfuric acid, sulfurous acid, thiocyanic acid and thiosulfuric acid.

The organic acid may be selected from the group consisting of acetic acid, C₂H₅COOH, C₃H₇COOH, C₄H₉COOH, (COOH)₂, CH₂(COOH)₂, C₂H₄(COOH)₂, C₃H₆(COOH)₂, C₄H₈(COOH)₂, C₅H₁₀(COOH)₂, fumaric acid, maleic acid, malonic acid, lactic acid, citric acid, tartaric acid, oxalic acid, ascorbic acid, benzoic acid, salicylic acid, phthalic acid, carbonic acid, formic acid, methanesulfonic acid, ethanesulfonic acid, camphoric acid, gluconic acid, L- and D-glutamic acid, pyruvic acid, L- and D-aspartic acid, trifluoroacetic acid, ranelic acid, gluconic acid, L- and D-glutamic acid, L- and D-aspartic acid, trifluoroacetic acid, ranelic acid, 2,3,5,6-tetrabromobenzoic acid, 2,3,5,6-tetrachlorobenzoic acid, 2,3,6-tribromobenzoic acid, 2,3,6-trichlorobenzoic acid, 2,4-dichlorobenzoic acid, 2,4-dihydroxybenzoic acid, 2,6-dinitrobenzoic acid, 3,4-dimethoxybenzoic acid, abietic acid, acetoacetic acid, acetonedicarboxylic acid, aconitic acid, acrylic acid, adipic acid, alphaketoglutaric acid, anthranilic acid, benzilic acid, arachidic acid, azelaic acid, behenic acid, benzenesulfonic acid, beta-hydroxybutyric acid, brassidic acid, capric acid, chloroacrylic acid, cinnamic acid, citraconic acid, crotonic acid, cyclopentane-1,2-dicarboxylic acid, cyclopentanecarboxylic acid, cystathionine, decanoic acid, erucic acid, ethylenediaminetetraacetic acid, fulvic acid, fumaric acid, gallic acid, glutaconic acid, glutaric acid, gulonic acid, glucosamine sulphate, heptanoic acid, hexanoic acid, humic acid, hydroxystearic acid, isophthalic acid, itaconic acid, lanthionine, lauric acid (dodecanoic acid), levulinic acid, linoleic acid (cis, cis-9,12-octadecadienoic acid), malic acid, m-chlorobenzoic acid, melissic acid, mesaconic acid, methacrylic acid, monochloroacetic acid, myristic acid, (tetradecanoic acid), nonanoic acid, norvaline, octanoic acid, oleic acid (cis-9-octadecenoic acid), ornithine, oxaloacetic acid, palmitic acid (hexadecanoic acid), p-aminobenzoic acid, p-chlorobenzoic acid, petroselic acid, phenylacetic acid, p-hydroxybenzoic acid, pimelic acid, propiolic acid, propionic acid, p-tert-butylbenzoic acid, p-toluenesulfonic acid, pyruvic acid, sarcosine, sebacic acid, serine, sorbic acid, stearic acid (octadecanoic acid), suberic acid, succinic acid, terephthalic acid, tetrolic acid, threonine, L-threonate, thyronine, tricarballylic acid, trichloroacetic acid, trimellitic acid, trimesic acid, tyrosine, ulmic acid and cylohexanecarboxylic acid.

All acids, which FDA has regarded as safe for use in compositions for oral intake, may be used in the present invention. Examples of suitable acids are mentioned in the following 25 table I:

TABLE I
Acids for making strontium salts

ACETIC ACID,
N-ACETYL-L-METHIONINE
ACONITIC ACID
ACRYLIC ACID-2-ACRYLAMIDO-2-METHYL PROPANE
SULFONIC ACID COPOLYMER
ADIPIC ACID
ALGINIC ACID
P-AMINOBENZOIC ACID
ANISIC ACID
ASCORBIC ACID
L-ASPARTIC ACID
D-ASPARTIC ACID
BENZOIC ACID
BORIC ACID
BUTTER ACIDS
BUTYRIC ACID
CHOLIC ACID
CINNAMIC ACID
CITRIC ACID
CYCLOHEXANEACETIC ACID
CYCLOHEXANECARBOXYLIC ACID
DECANOIC ACID
4-DECENOIC ACID
5-DECENOIC ACID
6-DECENOIC ACID
9-DECENOIC ACID
DEHYDROACETIC ACID
DESOXYCHOLIC ACID
2,4-DIHYDROXYBENZOIC ACID
3,7-DIMETHYL-6-OCTENOIC ACID
2,4-DIMETHYL-2-PENTENOIC ACID
(E)-2-DECENOIC ACID
EDTA, CALCIUM DISODIUM
(E)-2-HEPTENOIC ACID
(E)-2-NONENOIC ACID
(E)-2-OCTENOIC ACID
ERYTHORBIC ACID
ETHANESULFONIC ACID, 2-(1-(DIFLUORO-((TRI
FLUOROETHENYL)O
2-ETHYLBUTYRIC ACID
4-ETHYLOCTANOIC ACID
FATTY ACIDS
FOLIC ACID
FORMIC ACID
FUMARIC ACID
D-GLUCONIC ACID
L-GLUTAMIC ACID
D-GLUTAMIC ACID
GLUCOSAMINE SULPHATE
GLYCOCHOLIC ACID
HEPTANOIC ACID
HEXANOIC ACID
TRANS-2-HEXENOIC ACID
3-HEXENOIC ACID
HYDROCHLORIC ACID
4-HYDROXYBENZOIC ACID
1-HYDROXYETHYLIDENE-1,1-DIPHOSPHONIC ACID
3-HYDROXY-2-OXOPROPIONIC ACID
ISOBUTYRIC ACID
ISOVALERIC ACID
ALPHA-KETOBUTYRIC ACID
LACTIC ACID
LAURIC ACID
LEVULINIC ACID
LIGNOSULFONIC ACID
LINOLEIC ACID
L-MALIC ACID
MALIC ACID
2-MERCAPTOPROPIONIC ACID
METHACRYLIC ACID-DIVINYLBENZENE COPOLYMER
2-METHOXYBENZOIC ACID
3-METHOXYBENZOIC ACID
4-METHOXYBENZOIC ACID
TRANS-2-METHYL-2-BUTENOIC ACID
2-METHYLBUTYRIC ACID
3-METHYECROTONIC ACID
2-METHYLHEPTANOIC ACID
2-METHYLHEXANOIC ACID
5-METHYLHEXANOIC ACID
4-METHYLNONANOIC ACID
4-METHYLOCTANOIC ACID
3-METHYL-2-OXOBUTANOIC ACID
3-METHYL-2-OXOPENTANOIC ACID
4-METHYL-2-OXOPENTANOIC ACID
3-METHYLPENTANOIC ACID
4-METHYLPENTANOIC ACID
2-METHYL-2-PENTENOIC ACID
2-METHYL-3-PENTENOIC ACID
2 METHYL-4-PENTENOIC ACID
4-(METHYLTHIO)-2-OXOBUTANOIC ACID
2-METHYLVALERIC ACID
MONOCHLOROACETIC ACID--PROHIBITED
MYRISTIC ACID
NONANOIC ACID
NORDIHYDROGUAIARETIC ACID--PROHIBITED
9,12-OCTADECADIENOIC ACID (48%) AND 9,12,15-
OCTADECATRIENOIC ACID
OCTANOIC ACID
OLEIC ACID
OLEIC ACID, FROM TALL OIL FATTY ACIDS
2-OXOPENTANEDIOIC ACID
2-OXO-3-PHENYLPROPIONIC ACID
PALMITIC ACID
4-PENTENOIC ACID
PERACETIC ACID
PERIODIC ACID
PHENOXYACETIC ACID
PHENYLACETIC ACID
3-PHENYLPROPIONIC ACID
PHOSPHORIC ACID
POLYMALEIC ACID
PROPIONIC ACID
PYROLIGNEOUS ACID
PYROLIGNEOUS ACID, EXTRACT
PYRUVIC ACID
SALICYLIC ACID
SORBIC ACID
STEARIC ACID
SUCCINIC ACID
SULFURIC ACID
SULFUROUS ACID
TANNIC ACID
TARTARIC ACID, L
TAUROCHOLIC ACID
1,2,5,6-TETRAHYDROCUMINIC ACID
THIODIPROPIONIC ACID
L-THREONIC ACID
TRIFLUOROMETHANE SULFONIC ACID
UNDECANOIC ACID
10-UNDECENOIC ACID
N-UNDECYLBENZENESULFONIC ACID
VALERIC ACID
VANILLIC ACID

In one embodiment of the invention, the acid may be a non-chelator of strontium. In yet a further embodiment, the acid may be a monoprotic or a diprotic acid.

As mentioned above, the strontium salt for use according to the invention may be water soluble, having a water solubility of at least 0.1 g/l such as, e.g., in a range of from about 0.1 g/l to about 10 g/l, from about 0.2 g/l to about 5 g/l at room temperature exemplified e.g., by strontium citrate, strontium fumarate, strontium sulphate, strontium hydrogen phosphate, strontium tartrate and strontium oxalate, or in a range from about 1 g/l to about 200 g/l exemplified e.g., by strontium maleate, strontium glutamate, strontium aspartate, strontium pyruvate, strontium alpha-ketoglutarate, strontium malonate, strontium succinate etc., i.e., in a specific aspect of the invention the strontium salt has a water-solubility of at least 1 g/l, such as, e.g., at least 5 μl, at least 10 g/l, at least 20 gil, at least 30 g/l, at least 40 g/l, at least 50 g/l, at least 60 g/l, at least 70 g/l, at least 80 g/l, at least 90 g/l or at least 100 μl measured at room temperature of (20-25° C.).

However, as mentioned above, in a specific embodiment of the invention the strontium salts have a less pronounced water-solubility such as, e.g., at the most about 10 g/l such as, e.g., at the most about 5 g/l. To this end especially salts like e.g., strontium fumarate, strontium tartrate, strontium ranelate, strontium carbonate, strontium oxalate, strontium sulphate, strontium hydrogen phosphate and strontium citrate are used in a composition according to the invention.

Specific examples of strontium salts for use according to the invention are strontium chloride, strontium chloride hexahydrate, strontium citrate, strontium malonate, strontium succinate, strontium fumarate, strontium ascorbate, strontium L-aspartate, strontium D-aspartate, strontium L-glutamate, strontium D-glutmate, strontium tartrate, strontium glutarate, strontium glucosamine sulphate, strontium D-threonate, strontium L-threonate, strontium maleate, strontium methanesulfonate, strontium benzenesulfonate, and mixtures thereof.

Other examples of relevant acids for making strontium salts for use in a pharmaceutical composition may be found in WO 00/01692, which is hereby incorporated by reference.

The daily dose of strontium may be at least about 0.01 g, such as, e.g., at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as, e.g., from about 0.1 to about 2 g, from about 0.1 to about 1 g, from about 0.15 to about 0.5 g, from about 0.3 to about 2 g or from about 0.3 to about 1 g.

### Synthesis of Strontium Salts

Organic strontium salts of carboxylic acid anions can be synthesized by a number of different pathways. A conventional method for preparation of such organic strontium salts is to utilize the reaction between and organic acid and strontium hydroxide in an aqueous solution. This neutralization reaction of, e.g., fumaric acid and strontium hydroxide salt follows the following scheme:

Sr²⁺(aq)+2OH⁻(aq)+HOOCCHCHCOOH(aq)→Sr(OOCCHCHCOO)(aq)+2H₂O(1)

The suspension of dissolved strontium fumarate can then be induced to precipitate by sublimation of water and subsequent up-concentration of the salt. Crystals will slowly form and precipitate from the solution.

An alternative approach is to utilise the sodium or potassium salt of the appropriate carboxylic acid anion and strontium chloride. As all organic strontium salts will be less soluble than the highly soluble chloride salt, the organic strontium salt will precipitate under these conditions leaving NaCl and excess SrCl₂ in the solution. The equation below exemplifies this reaction scheme using as an example the reaction between SrCl₂ and sodium-fumarate.

Sr²⁺(aq)+2Cl⁻(aq)+2Na⁺(aq)+C₄H₂O₄²⁻(aq)→Sr(OOCCHCHCOO)(aq)+Cl⁻(aq)+Na⁺(aq)

The present inventors have found that different strontium salts requires different synthesis pathways, and for some strontium salts we have identified optimized synthesis and manufacturing procedures. Of particular relevance for the present invention, it has been found that synthesis of strontium salts of the di-carboxylic amino acids aspartate and glutamate (in either D- or L-form) is very difficult when following these conventional reaction pathways, and generally results in low yields and purity of the obtained crystalline salt. In order to facilitate large-scale manufacture of pure strontium salts of dicarboxylic amino acids to carry out the pharmaceutical use according to the present invention, the present inventors have studied various synthesis pathways of these particular strontium salts. Thus, it has surprisingly been found that synthesis of well defined and pure strontium glutamate in hexahydrate form is most convenient carried out with the free acid form of glutamate and strontium hydroxide and requires elevated temperatures, such as temperatures above 80° C., or more preferred 100° C. or even 120° C. or most preferred more than 130° C. (see example 7, where novel manufacturing procedures for synthesis of organic strontium salts at high temperature are described).

Furthermore, we have found that addition of small volumes of alcohol can accelerate the crystal-formation of dissolved aqueous organic strontium salts. Examples of these synthesis procedures for organic strontium salts of relevance for the treatment and/or prophylaxis of bone disease are provided in the examples herein.

### Pharmaceutical Compositions

The invention also relates to pharmaceutical composition comprising at least one strontium compound as described above. The pharmaceutical compositions according to the invention normally further comprise one or more physiologically acceptable excipients, i.e., a therapeutically inert substance or carrier.

The carrier may take a wide variety of forms depending on the desired dosage form and administration route.

The pharmaceutical composition comprising a compound according to the invention may be in the form of a solid, semi-solid or fluid composition. The composition is designed to release the active substance in the gastrointestinal tract, i.e., in a preferred aspect it is not intended for application to or absorption via the oral mucosa.

The solid composition may be in the form of tablets such as, e.g., conventional tablets, effervescent tablets, coated tablets, melt tablets or sublingual tablets, pellets, powders, granules, granulates, particulate material, solid dispersions or solid solutions.

In one embodiment of the invention, the pharmaceutical composition may be in the form of a tablet. The tablet may be coated with a coating that enables release of at least part of the salt in proximal part of the small intestine, such as e.g., the duodenum and/or the proximal jejunum such as at least 50% w/w, at least 60% w/w, at least 65% w/w, at least 70% w/w, at least 80% w/w or at least 90% w/w of the total amount of the salt contained in the tablet.

The tablet may have a shape that makes it easy and convenient for a patient to swallow. The tablet may thus e.g., have a rounded or a rod-like shape without any sharp edges. Furthermore, the tablet may be designed to be divided in two or more parts.

A semi-solid form of the composition may be a paste, a gel or a hydrogel.

The fluid form of the composition may be a solution, an emulsion including nano-emulsions, a suspension, a dispersion, a liposomal composition, a spray, a mixture, a syrup or an elixir.

Other suitable dosages forms of the pharmaceutical compositions according to the invention may be capsules, sachets, troches, devices etc.

The pharmaceutical compositions may be prepared by any of the methods well known to a person skilled in pharmaceutical formulation.

The pharmaceutically acceptable excipients may be e.g., fillers, binders, disintegrants, diluents, glidants, solvents, emulsifying agents, suspending agents, stabilizers, enhancers, flavors, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook. In those cases, where the pharmaceutical composition is intended for controlled release of the Sr containing compound, it may also comprise release controlling agents such as, e.g., material normally used in the formulation of matrix tablets (e.g., cellulose derivatives like hydroxypropyl methylcellulose and the like). Alternatively, the composition may be coated with a controlled release coating such as an enteric coating or a film coating. A suitable coating may be a substantially water-insoluble but water-permeable coating.

As mentioned above, the invention relates to a controlled release pharmaceutical composition for oral use. The composition may be in the form of a tablet, a capsule, a multiparticulate form, or a unit dose packet such as a sachet.

The term “tablet” is intended to embrace compressed tablets, coated tablets, matrix tablets, osmotic tablets, and other forms known in the art.

The term “capsule” is intended to embrace hard and soft capsules, in which the shell of the capsule disintegrates after ingestion to release its content.

The term “multiparticulate” is intended to embrace a dosage form comprising a multiplicity of particles and/or granulates whose totality represents the intended therapeutically useful dose. The particles generally are of a diameter from about 50 microns to about 0.3 cm, with a preferred range of 100 μm to 1 mm. Multiparticulates represent a suitable embodiment for use in scaling dosage forms release because they are amenable according to the weight of an individual subject (e.g., a mammal such as a human).

In a further aspect, this invention provides a process for preparing controlled or delayed release dosage forms of a strontium salt e.g., comprising the steps of mixing or granulating the strontium salt together with one or more pharmaceutically acceptable excipients (selected from the group consisting of fillers, binders, disintegrants, release rate modifier, etc.) to obtain a powder blend that can be further processes into e.g., matrix pellets or tablets or into pellets or tablets that are provided with a controlled release polymer coating controlling the release of the strontium salt.

In the case of delayed release embodiments, the dosage form can take the same forms as above, provided that the dosage form delivers the majority of its strontium salt to regions of the gastrointestinal tract distal to the duodenum. A variety of dosage form embodiments and/or structures may be used to achieve this goal, i.e., multiparticulates, beads, pellets or other particle dosage forms that may be multiply loaded into a gelatin capsule or may be compressed into a tablet.

The controlled or delayed release dosage forms of this invention can be widely implemented. Different embodiments include e.g., matrix systems, in which the strontium salt is embedded or dispersed in a matrix of another material that serves to retard the release of the active substance into an aqueous environment (i.e., the lumen fluid of the GI tract). When the strontium salt is dispersed in a matrix of this sort, release of the drug takes place principally from the surface of the matrix.

Thus the active substance is released from the matrix surface after it has diffused through the matrix or when the surface of the composition erodes and thus exposes the active substance. In some embodiments, both mechanisms can operate simultaneously. The matrix systems may be large, i.e., tablet sized (about 1 cm), or small (<0.3 cm). The system may be a single unit or multiple units, which are administered substantially simultaneously, or may comprise a plurality of particles, referred to herein as a multiparticulate. A multiparticulate can have numerous formulation applications. For example, a multiparticulate may be used as a powder for filling a capsule shell, or used per se for mixing with food to increase palatability.

Slowly-hydrating materials may also be used to give the desired release rate. The multiplicity of variables affecting release of the active substance from matrices permits flexibility in the design of compositions of different materials, sizes, and release times. Examples of modifications of strontium ion release profiles are within the scope of this invention.

A specific embodiment of the invention relates to a matrix multiparticulate comprises a plurality of strontium salt-containing particles, each particle comprising a mixture of the strontium salt with one or more appropriate pharmaceutically acceptable excipient selected to form a matrix capable of limiting the dissolution rate of the strontium salt into an aqueous medium. The matrix materials useful for this embodiment are generally water-insoluble materials such as waxes, cellulose, or other water-insoluble polymers. If needed, the matrix materials may optionally be formulated with water-soluble materials, which can be used as binders or as permeability modifying agents. Matrix materials useful for the manufacture of these dosage forms include microcrystalline cellulose such as Avicel (registered trademark of FIVIC Corp., Philadelphia, Pa.), including grades of microcrystalline cellulose to which binders such as hydroxypropyl methyl cellulose have been added, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble binders or release modifying agents which can optionally be formulated into the matrix include water-soluble polymers such as hydroxypropyl cellulose (HPC), hydroxypropyl methyl cellulose (HPIVIC), Methyl cellulose, poly (N-vinyl pyrrolidinone) (PVP), poly(ethylene oxide) (PEO), poly(vinyl alcohol) (PVA), xanthan gum carrageenan, and other such natural and synthetic materials. In addition, materials that function as modifying agents include water-soluble materials such as sugars or salts. Preferred water-soluble materials include lactose, sucrose, glucose, and mannitol, as well as HPC, HPMC, and PVP.

A suitable process for manufacturing matrix multiparticulates is the extrusion/spheronization process. For this process, the active substance is wet massed with a binder, extruded through a perforated plate or die, and placed on a rotating disk.

The extrudate ideally breaks into pieces, which are rounded into spheres, spheroids, or rounded rods on the rotating plate.

A further preferred process for manufacturing matrix multiparticulates is the preparation of wax granules. In this process, a desired amount of the active substance is stirred with a wax to form a homogeneous mixture, which is cooled and then forced through a screen to form granules. Suitable matrix materials are waxy substances such as, e.g., hydrogenated castor oil and carnauba wax and stearyl alcohol.

A further process for manufacturing matrix multiparticulates involves using an organic solvent to aid mixing of the active substance with the matrix material. This technique can be used when it is desired to utilize a matrix material with an unsuitably high melting point that, if the material were employed in a molten state, would cause decomposition of the drug or of the polymeric matrix material. Alternatively, the active substance and matrix material may be combined with a solvent to dissolve the matrix material and the resulting solution (which may contain solid drug particles) is e.g., spray dried to form the particulate dosage form. This technique is preferred when the matrix material is a high molecular weight synthetic polymer such as any cellulose ether or cellulose ester. Solvents typically employed for the ethanol, isopropanol, ethyl acetate, and mixtures process include acetone, of two or more.

Once formed, the matrix multiparticulates may be blended with compressible excipients such as lactose, microcrystalline cellulose, calcium phosphate, and the like and the blend is compressed to form a tablet. Disintegrants such as sodium starch glycolate or crosslinked poly(vinyl pyrrolidone) are also usefully employed. Tablets prepared by this method disintegrate when placed in an aqueous medium (such as the GI tract), thereby exposing the multiparticulate matrix, which releases the strontium salt and/or the ionic form of free strontium from the composition.

A further embodiment of a matrix system has the form of a hydrophilic matrix tablet containing the active substance and an amount of hydrophilic polymer sufficient to provide a useful degree of control over the dissolution of the strontium salt. Hydrophilic polymers useful for forming the matrix include hydroxypropylmethyl cellulose (HPMC), hydroxypropyl cellulose (HPC), poly(ethylene oxide), poly(vinyl alcohol), xanthan gum, carbomer, carrageenein, and zooglan. A suitable material is HPMC. Other similar hydrophilic polymers may also be employed. Using a lower molecular weight polymer may increase the dissolution rate. The dissolution rate may also be controlled by the use of water-soluble additives such as sugars, salts, or soluble polymers. Examples of these additives are sugars such as lactose, dextrose, cyclo-dextrose, sucrose, or mannitol, salts such as NaCl, KCl, NaHCO3, and water soluble polymers such as PNVP or PVP, low molecular weight, HPC or HIVIPC or methyl cellulose. In general, increasing the fraction of soluble material in the formulation may increase the release rate. A matrix tablet typically comprises about 20 to 90% by weight of the active substance and about 10 to 80% by weight of polymer.

A suitable matrix tablet comprises, by weight, about 50% to about 80% the active substance about 15% to about 35% HPMC, 0% to about 35% lactose, 0% to about 15% PVP 0% to about 20% microcrystalline cellulose, and about 0.25% to about 2% magnesium stearate.

The matrix systems as a class often exhibit non-constant release of the drug from the matrix. This result may be a consequence of the diffusive mechanism of drug release, and modifications to the geometry can be used to make the release rate of the drug more constant as detailed below.

In a further embodiment, a matrix tablet may be coated with an impermeable coating and an orifice (for example, a circular hole or a rectangular opening) is provided by which the content of the tablet is released.

In a suitable embodiment a tablet or capsule is coated with an impermeable material on part of its surface, e.g., on one or both tablet faces, or on the tablet radial surface.

The dosage form may be coated with a coating that modulates the release of the active substance. By “impermeable material” is meant a material having sufficient thickness and impermeability to the active substance such that no significant transport thereof can take place through the material during the time scale of the intended drug release (i.e., several hours to about a day). Such a coating can be obtained by selecting a coating material with a sufficiently low diffusion coefficient for the active substance and applying it sufficiently thickly.

Materials for forming the impermeable coating of these embodiments include substantially all materials in which the diffusion coefficient of the active substance is suitable. Preferred coating materials include film-forming polymers and waxes. Especially preferred are thermoplastic polymers, such as poly(ethylene-co-vinyl acetate), poly(vinyl chloride), ethylcellulose, and cellulose acetate. These materials exhibit the desired low permeation rate of the active substance when applied as coatings.

A further controlled release matrix system comprises the active substance dispersed in a hydrogel matrix. This embodiment differs from the hydrophilic matrix tablet discussed above in that the hydrogel of this embodiment is not a compressed tablet of erodible granular material, but rather a monolithic polymer network. As known in the art, a hydrogel is a water-swellable network polymer. Hydrogels are preferred materials for matrix devices because they can absorb or be made to contain a large volume fraction of water, thereby permitting diffusion of solvated drug within the matrix.

Diffusion coefficients of drugs in hydrogels are characteristically high, and for highly water-swollen gels, the diffusion coefficient of the drug in the gel may approach the value in pure water. This high diffusion coefficient permits practical release rates from relatively large devices (i.e., it is not necessary to form microparticles). Preferred materials include hydrophilic vinyl and acrylic polymers, polysaccharides such as calcium alginate, and poly(ethylene oxide). Especially suitable are poly(2-hydroxyethyl-methacrylate), poly(acrylic acid), poly(methacrylic acid), poly(N-vinyl pyrrolidinone), poly(vinyl alcohol) and their copolymers with each other and with hydrophobic monomers such as methyl ethacrylat el vinyl acetate, and the like. Also preferred are hydrophilic polyurethanes containing large poly(ethylene oxide) blocks. Other preferred materials include hydrogels comprising interpenetrating networks of polymers, which may be formed be addition or by condensation polymerization, the individual monomers components may comprise hydrophilic and hydrophobic groups.

Other coating materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary bed coater. If desired, the permeability of the coating may be adjusted by blending of two or more materials. A particularly useful process1 for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water-soluble membrane additives are leached out of the membrane, leaving pores which facilitate release of the drug. The membrane coating can also be modified by the addition of plasticizers, as known in the art.

Above are mentioned specific examples of the amounts of compounds administered. However, it will be understood that the amount of the compounds actually administered will be determined by a physician in light of the relevant circumstances including the condition to be treated, the choice of compounds to be administered, the age, weight, and response of the individual patient, the severity of the patient's symptoms and/or signs, and the chosen route of administration. While the present compounds are preferably administered orally, the compounds may also be administered by any other suitable route.

### Treatment of Males

Contrary to popular belief, osteoporosis is not just a disease of women. Males are not as resistant to osteoporosis as once thought, and the classical age-related increase in fractures seen in women is also evident in men. One of the main reasons why osteoporosis is not as common in males as in women is the larger skeleton of the males. Other factors include the shorter life expectancy, later onset and slower progress of bone loss in men, and the absence of rapid bone loss that affects women as a result of cessation of endogenous oestrogen production at the menopause.

However, as understanding of the pathophysiology of the disease has increased in recent years, it's been recognized that male osteoporosis represents an important public health issue. In the United States alone, up to 5 million men suffer from osteoporosis, and their number is rising. Almost 30-40% of patients develop so-called ‘idiopathic’ osteoporosis at a young age, in the absence of any detectable cause, whereas others have multiple evident secondary reasons for bone loss, including glucocorticoid excess, hypogonadism, alcohol abuse, smoking, renal tubular disease with calcium wasting, or other liver or bowel diseases.

Accordingly, the invention relates to a method for the treatment and/or prophylaxis of a cartilage and/or bone disease and/or conditions resulting in a dysregulation of cartilage and/or bone metabolism in a male subject, such as e.g., osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues and for weight gain, the method comprising administering a Sr salt in an amount and frequency that may give a daily dose of from about 0.25 to about 1.5 g ionic free Sr²⁺, such as, e.g., from about 0.30 g to about 1.5 g, from about 0.40 g to about 1.40 g, from about 0.50 g to about 1.30 g, from about 0.60 g to about 1.20 g, from about 0.60 g to about 1.0 g or from about 0.60 g to about 0.8 g.

The Sr salt may be administered orally, and may be contained in a pharmaceutical composition as defined above.

### Prophylaxis

Some of the drugs used today for the treatment of diseases and conditions affecting metabolism and/or structural integrity of bone and/or cartilage may have a therapeutic effect on the condition, but at the same time many of the drugs used are associated with severe side effects. An example of a group of drug substances with severe side effects are the bisphosphonates, which appear to have detrimental side effects, such as, e.g., the potential of inhibiting bone formation as well as resorption and poor absorption via oral administration. Furthermore, they are known to cause G.I. irritation and to have extremely long half-lives in bone. Therefore, the subject in need of treatment potentially should have a minimal exposure to such compounds. Accordingly, such drug substances are not suitable for prophylactic treatment.

As there are no known side effects associated with the administration of strontium in the doses suitable for prophylaxis, strontium will probably be very useful for the prevention of cartilage and/or bone conditions. Accordingly, the invention relates to a method for the treatment and/or prophylaxis of a cartilage and/or bone disease and/or conditions resulting in a dysregulation of cartilage and/or bone metabolism in a mammal, such as e.g., a human female or male adult, adolescent or child, such as, e.g., osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues and for weight gain, the method comprising administering a Sr containing compound.

The measurement of BMD, bone mineral density, or other forms of radiographic assessment of bones or joints can be used to establish or confirm a diagnosis of diseases and conditions affecting metabolism and/or structural integrity of cartilage and/or bone, such as, e.g., osteoporosis and osteopenia. The BMD value may also be used for determining whether a prophylactic treatment should be initiated.

Several techniques are available to measure BMD non-invasively. Bone densitometry is the best available method for diagnosing osteoporosis and osteopenia and other bone conditions, and for identifying subjects at risk for developing a bone condition. In bone densitometry the amount of bone mineral present is measured, which is an important determinant of bone strength. The traditional bone densitometry methods are based on X-ray absorptiometry, such as, e.g., dual-energy x-ray absorptiometry, single-energy X-ray absorptiometry and radiographic absorptiometry. In addition to these approaches, qualitative computed tomography, which utilizes a computed tomography scan, can be used to calculate BMD. Another bone densitometry method is quantitative ultrasound, which is fairly inexpensive, portable and radiation free. Here the bone part to be measured is positioned between two ultrasound transducers, and bone mass is determined by the transmission of sound waves passing through the bone; the fewer that pass, the denser the bone.

In order to standardize values from different densitometers, and to give a value that easily can be used for diagnosis, the BMD value may be used to calculate values called the T-score and the Z-score.

The T-score is considered the most clinically relevant value, and describes the subject's BMD relative to the mean BMD for young adult normal women or men, respectively, expressing the difference as number of standard deviations (SDs). In the case of females, if the T-score of a subject is less than 1 SD below the mean of young adult normal women, the BMD is considered normal. For each SD below the mean bone mass of young adult normal women, the risk of fracture increases by approximately 1.5 to 3 fold, and below 2 SDs, it increases exponentially.

The Z-score compares the subject's BMD to the mean BMD for males or females having the same age as the subject. Among older adults, however, a low BMD is common, so comparison with age-matched norms may be misleading, i.e., an 80-year-old subject may have a Z-score that compares favorably with age-matched controls, but nevertheless, like the average patient in this age group, the subject may be at risk of experiencing fracture(s).

In a method according to the invention for preventing a cartilage and/or a bone condition, subjects who are at risk of developing such a condition may be identified by calculating the subjects' T-scores. Accordingly, the present invention relates to a method wherein the subject is a female having a bone mineral density, BMD, of more than 1 SD below the adult female mean.

In another method the Z-score is calculated, i.e., the present invention relates to a prophylactic method, wherein the subject is a female having a BMD below the female mean for women of the same age. If the female belongs to an age-group wherein the average females may have a higher risk of bone fracture, a prophylactic treatment may be considered, even though the female has a BMD at or above the value of the female mean for women of the same age.

The invention also relates to a prophylactic method as described above, wherein the subject is a male having a BMD of more than 1 SD below the adult male mean.

Furthermore, the invention also relates to a method wherein the subject is a male having a BMD below the adult male mean for men of the same age. If the male belongs to an age-group wherein the average males may have a higher risk of bone fracture, a prophylactic treatment may be considered, even though the male has a BMD at or above the value of the male mean for men of the same age.

The invention also relates to a prophylactic method, wherein the subject is a 20 year or older such as, e.g., 25 years or older, 30 years or older, 35 years or older, 40 years or older, 45 years or older, or 50 years or older female.

Another way of assessing the status of bone and cartilage is provided by dynamic biochemical markers, which reflect the turnover of either cartilage or bone. In comparison to BMD or other similar static measurements, which provide a measure of the current status of bone and/or cartilage, a specific biomarker can provide a measure of the current turnover of the tissue from which the marker is derived. This provides a dynamic monitoring of therapeutic effects and it also enables a prediction of progression of a disease or condition affecting bone and/or cartilage turnover. As an example it has been demonstrated in several studies that a specific marker of bone resorption, C-telopeptide derived collagen type I fragments, CTX, provides a BMD independent predictor of subsequent fracture risk, with similar potency as BMD measurements alone. Furthermore, the measure of fracture risk provided by CTX measurements is additive to the measure provided by BMD determinations, and thus individuals with both a low BMD, and a high bone turnover as indicated by elevated CTX levels are more at risk for sustaining skeletal fracture than individuals with only elevated BMD or decreased CTX. Similar data has been obtained with a specific marker of cartilage derived collagen type II fragments, CTX-II. Accordingly subjects at risk of developing pathological deterioration of bone and/or cartilage may be defined by measurement of specific biomarkers of either bone or cartilage metabolism. Similar to BMD measurements, the biomarker measurements may be expressed in T-scores or Z-scores related to relevant reference populations, or the values may simply be expressed relative to pre-defined cut-of levels.

The invention also relates to a prophylactic method as described above, wherein the subject is a male having a biomarker level of a bone resorption marker such as CTX or NTX of more than 1 SD above the adult male mean.

Furthermore, the invention also relates to a method wherein the subject is a male having a bone resorption marker such as CTX or NTX above the adult male mean for men of the same age. If the male belongs to an age-group wherein the average males may have a higher risk of bone fracture, a prophylactic treatment may be considered, even though the male has a bone resorption marker such as CTX or NTX below the mean levels of the given marker for men of the same age.

The invention also relates to a prophylactic method, wherein the subject is a 20 year or older such as, e.g., 25 years or older, 30 years or older, 35 years or older, 40 years or older, 45 years or older, or 50 years or older female.

Furthermore, the invention relates to a combined use of BMD measurement and one or more biomarkers for definition and/or prediction of individuals of risk for progression of a disease or condition affecting bone and/or cartilage turnover.

Estrogen plays an important role in bone health, as estrogen protects the bones by preventing the skeletal system from elevation in bone turnover, which results in imbalance between formation and resorption of bone and subsequent skeletal deterioration. When the level of estrogen decreases after menopause, more bone is resorbed than is built. Women who do not take any form of medication preventing bone loss may lose as much as 3% to 5% of their bone mass in each of the 5 years following menopause, and by e.g., age 70, the bones can weigh 30% to 50% less than before menopause.

Accordingly, even though a female at the onset of menopause may have a BMD level and/or level of specific biomarkers of bone and/or cartilage turnover within the normal range (i.e., as defined by the T score), it may be beneficial to initiate a treatment for preventing the development of a bone condition in the future. Thus, the invention relates to a method as described above, wherein the subject is a peri-menopausal female or a female with recent onset of menopause.

Furthermore, the invention relates to a method, wherein the subject is a female who is about 6 months or more beyond the onset of menopause.

The invention also relates to a method for prophylaxis of a cartilage and/or bone condition in males, wherein the subject is a 20 year or older such as, e.g., 25 years or older, 30 years or older, 35 years or older, 40 years or older, 45 years or older, 50 years or older, 55 years or older, 60 years or older, 65 years or older, or 70 years or older male.

### Secondary Osteoporosis

Even though 90% of all osteoporosis cases are idiopathic primary osteoporosis, there also exists a need for preventing and/or treating secondary osteoporosis, which is the result of an identifiable disease process or agent. Accordingly, the invention relates to a method for treating and/or preventing secondary osteoporosis in a subject, the method comprising administering an effective amount of a Sr salt to the subject.

Secondary osteoporosis may be induced by endocrine diseases and/or metabolic causes, such as, e.g., hypogonadism, hypercortisolism, hyperprolactinemia, anorexia nervosa, mastocytosis, porphyria, diabetes mellitus type I, primary or secondary hyperparathyroidism, hyperthyroidism, acromegaly, Cushing's syndrome, acidosis, Guacher's disease, hemochromatosis, androgen insensitivity and pregnancy.

The secondary osteoporosis may also be induced by nutritional conditions, such as, e.g., malabsorption, malnutrition, chronic hepatic disease, vitamin D deficiency, calcium deficiency, resections of parts of the gastrointestional tract (e.g., gastrectomy), smoking and alcohol abuse.

The administration of certain drug substances may also lead to secondary osteoporosis. Examples of such drugs substances are e.g., corticosteroids (including inhaled corticosteroids), heparin, anti-epileptic drugs (e.g., phenyloin), gonadotrophin releasing hormone analogs, loop diuretics, phenobarbital, anti-neoplastic agents/immunosuppressants (e.g., methotrexate and cyclosporin), thyroid hormones, depo-medroxyprogesterone acetate, calcineurin-calmodulin phosphatase inhibitors such as, e.g., Tacrolimus, and aromatase inhibitors, such as Formestane, Exemestane, Aminoglutethimide, Fadrozole, Rogletimide, Anastrozole, Letrozole and Vorozole.

A disturbed collagen metabolism and/or diseases of the connective tissue may also be the cause of secondary osteoporosis. Examples of such disorders are e.g., osteogenesis imperfecta, homocysteinuria, rickets, Ehlers-Danlos syndrome and Marfan's syndrome.

Bone marrow diseases such as, e.g., myeloma, thalassemia and leukemia may also be the cause of secondary osteoporosis. Also rheumatologic/inflammatory diseases such as, e.g., systemic lupus erythematosus, ankylosing spondylitis and rheumatoid arthritis may cause secondary osteoporosis.

In children and adolescents, causes of secondary osteoporosis include juvenile arthritis, juvenile rheumatoid arthritis, juvenile chronic arthritis, childhood malignancy, neuromuscular diseases such as cerebral palsy, spina bifida and muscular dystrophy, familial dysautonomia, fibrous dysplasia, juvenile Paget's disease (osteoprotegerin deficiency), familial idiopathic bone pain and isolated hyperphosphatasemia. Other potential causes of secondary osteoporosis in children and adolescents are excessive exercise, amenorrhea, dermatomyositis, asthma, inflammatory bowel disease, such as Crohn's disease, and muscular dystrophy

Other general causes of secondary osteoporosis may be hypophosphatasis, immobilization, cystic fibrosis, renal insufficiency, hypercalciuria, chronic obstructive pulmonary disease, mastocytosis, depression, spinal cord injury, sarcoidosis, malignancy, lymphoplasmacytoid lymphoma, organ transplantation and surgical as well as chemical castration of males (including, but not limited to the use of anti-androgens and/or gonadotropin releasing hormone analogues).

### Prophylaxis of Secondary Osteoporosis

As mentioned above, some drugs may be the cause of secondary osteoporosis. In order to prevent the development of drug induced secondary osteoporosis in a subject, it may be beneficial to administer a prophylactic amount of Sr as part of the same treatment regimen as the administration of the drug substance.

Thus, the invention relates to a method for preventing drug induced secondary osteoporosis in a subject, the method comprising administering to the subject a prophylactic amount of a Sr salt before, during or after treatment of the subject with the drug substance that induces osteoporosis.

The administration may take place substantially simultaneously with administration of the drug substance that induces osteoporosis, and the Sr salt and the drug substance that induces osteoporosis may be contained in the same pharmaceutical composition.

Accordingly, the invention relates to a pharmaceutical composition comprising a Sr salt and a drug substance that induces osteoporosis together with a pharmaceutically acceptable excipient.

The Sr salt and the drug substance may also be administered simultaneously in separate, co-administered compositions. When two separate formulations are being co-administered, each formulation, especially those for use by the oral route, may be color-coded or otherwise easily identifiably labeled in order to avoid confusion by the subject or physician.

The invention also relates to a kit, comprising the one or more pharmaceutical compositions together with instructions for administration.

### Other Aspects of the Invention

In some embodiments of the invention, the inventors have found that it is preferable if ranelate, if present at all, may be present in an amount of less than 5% w/w of the total amount of strontium.

As mentioned above, use of a composition or kit according to the invention may lead to improved fracture healing after traumatic or atraumatic fracture, where the fracture e.g., may be one of the following traumatic or atraumatic fractures: fracture to the distal radius, such as e.g., a Colle's fracture or a Smiths fracture, a fracture of the femur, such as e.g., the proximal femur, such as e.g., a cervical fracture, a trochanteric fracture or a subtrochanteric fracture.

The improved fracture healing may be defined in terms of reduction of the time a patient will require a plaster, reduction of the time to healing as defined on a X-ray, reduction in the time to fracture stability, improvement of callus formation as viewed by X-ray, reduction in time before appearance of callus formation as viewed by X-ray and/or reduction in time for regaining full or near-full mobility or physical activity level.

Other embodiments of the invention appear from the appended claims. The details and particulars described above and below and relating to the compounds and compositions according to the invention apply mutatis mutandis to the other aspects of the invention.

Other embodiments of the invention appear from the appended claims. The details and particulars described above and relating to the compounds and compositions according to the invention apply mulatis mutandis to the other aspects of the invention.

The invention is further illustrated in the examples that are not intended to limit the invention in any way.

### EXAMPLES

### Example 1

### General Method for Preparation of Crystalline Salts of Strontium by Precipitation from Dissolved Strontium Chloride and Dissolved Sodium Salts of the Appropriate Carboxylic Anions

In a glass-beaker of 100 mL volume, 5 g of the sodium salt of the carboxylic acid was dissolved in a small volume of water that was slightly heated at temperatures not greater than 30-50° C. The final volume was 25-50 mL. In another beaker 10 g of SrCl₂ (SrCl₂ hexahydrate, Sigma-Aldrich 43, 966-5) was dissolved in 100 mL of water. This latter solution was slowly decanted into the first solution of the dissolved sodium salt. The transfer continued until an initial cloudiness was observed, which resulted in a total volume of 50-100 mL. The solution was allowed to rest at room temperature (22-24° C.) for several days until significant amounts of crystallized precipitate of the organic strontium salt appeared.

The reaction that proceeds is exemplified by the reaction between strontium ions and sodium fumarate (reaction schemes (a) and (b)):

NaOOCHCHCOONa(s)+H₂O(l)→⁻OOCCHCHCOOH(aq)+2Na⁺(aq)+OH⁻(aq) (a)

⁻OOCCHCHCOOH(aq)+Sr²⁺(aq)→Sr(OOCCHCHCOO(aq)+H⁺(aq) (b)

In order to accelerate the crystallisation, we have found that addition of small volumes of ethanol, such as from 5-10 vol/vol % to 50-60% vol/vol induces a significant acceleration of the precipitation of the desired strontium salt. Addition of ethanol is of special importance in the synthesis of strontium salts with solubility exceeding 2 g/l at room temperature (22-24°), and will thus provide a substantial benefit for the synthesis of strontium salts of L-aspartate, L-glutamate and lactate. In order to reach the required product within a short period, it was essential to observe an initial crystallisation or an initial dimness in the solution right from the first stage.

After the precipitation, the solution was filtered on a Büchner funnel using a suction flask and the crystals were flushed in small volumes of ethanol. Crystals of some of the salts were very soluble, so in order to improve the yield of crystals, the solution was allowed to rest longer, such as at least 30-60 min. Repeated crystallisation resulted in yields of approx. 50%. Strontium salts of L-aspartate and of lactate were very soluble, with solubility exceeding 25 g/l in water at room temperature.

The lactate and L-glutamate salts of strontium were precipitated from solutions with an excess of strontium chloride and large crystals of the lactate salt were achieved by slow evaporation of the solvent.

### Example 2

### General Method for Preparation of Crystalline Salts by Neutralisation of Carboxylic Acids with Strontium Hydroxide

A small amount of the organic acid proper (0.75-3 g, see table below) was dissolved in water by heating to temperatures between 30° C.-50° C. Then, strontium hydroxide (Sigma Aldrich, Sr(OH)₂*8H₂O, MW 265.71, CAS no. 1311-10-0, approx. 10 g/L) was slowly added. Then, a magnetic stirring rod was added and the stirring and gentle heating (i.e., 30-50° C.) of the suspension was started. After some time, the solution clarifies and all the solid material dissolves. The heating is maintained, and after three hours of incubation, the solution is filtered while hot on a Büchner funnel. Very small amounts of impurities were left in the filter.

The filtrate was subsequently allowed to cool at room temperature overnight, which resulted in growth of fine-powdered crystals of the desired strontium salt. Further purifications of the salts can be performed by repeated re-crystallizations (table 2).

TABLE 2
Amounts of start reagent used for organic strontium salt synthesis and recoveries
in the synthesis of eight specific organic strontium salts following the general
reaction pathway with free-acid forms of the anion, and strontium hydroxide.

| Strontium Salt of | Sr(OH)₂ | Free | Amount | | Melting | | | Crystal |
|---|---|---|---|---|---|---|---|---|
| (free acid used): | *8H₂O | Acid | Obtained | Recovery* | Temp. | | Solubility | Structure |
| Fumarate¹ | 2.044 g | 1.140 g | 0.999 g | 99% | >380° | C. | Yes | No |
| α-ketoglutarate² | 2.017 g | 1.441 g | 0.828 g | 72% | >380° | C. | Yes | No |
| succinate | 2.098 g | 1.177 g | 0.958 g | 92% | 230° | C. | Yes | Yes |
| L-Ascorbate³ | 2.094 g | 1.805 g | 2.005 g | 15% | >380° | C. | Yes | No |
| L-Glutamate | 2.017 g | 1.453 g | 0.175 g | 15% | >380° | C. | Yes | Yes |
| Citrate | 2.057 g | 1.918 g | 1.123 g | 48% | >380° | C. | Yes | Yes |
| D-Aspartate | 2.190 g | 1.316 g | 0.167 g | 14% | >380° | C. | No | No |
| Tartrate | 2.070 g | 1.502 g | 2.005 g | 129% | >380° | C. | Yes | Yes |

Notes
*Recovery calculated in % of the strontium content in Sr(OH)₂ *8H₂O.
¹Fumaric acid is insoluble in water, and ethanol is added to the suspension until complete solubilization is achieved. The synthesis is continued with this material.
²The strontium-AKG salts has a slight brownish appearance
³In addition to the indicated amounts of strontium hydroxides and L-ascorbate an additional 4.087 g SrCl₂*6H₂O solubilized in water is added to the reaction mixture.

### Example 3

### Determinations of Solubility of Organic Strontium Salts

### Synthesis of Strontium Salts

The great majority of strontium salts could be obtained by reacting the sodium salt of the organic acid with strontium chloride following the general synthesis method described in example A. However, strontium citrate, strontium tartrate, strontium succinate and strontium α-ketoglutarate for the solubility investigations was obtained by synthesis from the free acid forms of the carboxylic acid and strontium hydroxide as described in example 2. Strontium glutamate was obtained as described in example 4, using an incubation temperature of 100° C. for obtaining pure and homogeneous hexahydrate crystals of strontium glutamate. Detailed investigations of solubility were carried with the strontium salts listed in table 3 below:

TABLE 3
Overview of strontium salts used in investigation of solubility.

| Strontium Salt | MW | % Sr |
|---|---|---|
| Sr-ranelate (*7H₂O) | 639.6 | 27.4 |
| SrCl₂ (*6H₂O) | 266.6 | 32.9 |
| Sr-fumarate (*6H₂O) | 309.7 | 28.3 |
| Srl-glutamate (*6H₂O) | 340.7 | 25.7 |
| Sr-α-ketoglutarate (*6H₂O) | 339.7 | 25.8 |
| Sr-aspartate (*3H₂O) | 272.7 | 32.1 |
| Sr-succinate (*6H₂O) | 311.7 | 28.1 |
| Sr-ascorbate (*6H₂O) | 545.8 | 16.1 |
| Sr-malenate (*6H₂O) | 309.7 | 28.3 |
| Sr-malonate (anhydrous) | 189.7 | 46.2 |
| Sr-pyruvate (*6H₂O) | 369.7 | 23.7 |
| Sr-tartrate (*6H₂O) | 343.7 | 25.5 |
| Sr-citrate (*6H₂O) | 749.1 | 35.1 |

MW indicates the molecular weight of the homogeneous crystalline form of the salt with the indicated amount of crystal water and % Sr gives the molar percentage that strontium constitutes of this crystalline form

The solubility of the organic carboxylic acid strontium salts, were measured in water. The solubility of these salts was also measured as a function of temperature. This was performed by incubating the saturated solutions of the salts in temperature controlled incubators. Furthermore the solubility of the salts was studied in pure distilled water as well as a 0.05 M ammonium carbonate buffered solutions, with a physiological pH of 7.5.

The buffered solutions were immersed into a bath of water temperature controlled at either room temperature (22-24° C.), at 30° C. or at 40° C. The test tubes were stirred and the solutions were subsequently incubated in an incubator with constant temperature for 24 hours. In order to eliminate any reminiscent strontium chloride influence on the determination of solubility, all the precipitate was collected at the bottom of the test tubes and the solutions above the precipitate were carefully removed and substituted by fresh solutions. After substitution of the solutions, the test tubes were stirred again and allowed to rest for another 24 hours. From these solutions, the dissolved proportions of the strontium salt were collected in volumes of 1 mL at the specified temperature. The solutions were diluted to 50 mL before analysis by Flame Atomic Absorption Spectrometry (F-AAS). Before subsequent series of sampling, the solutions were equilibrated at the next temperature for 24 hours.

### Analysis of Strontium by Flame Atomic Absorption Spectrometry F-AAS and ICP-MS

Two methods were used for quantification of strontium in solutions: Flame Atomic Absorption Spectrometry (F-AAS), and the more sensitive inductively-coupled-plasmamass spectrometry (ICP-MS). For most investigations, the F-AAS method had sufficient sensitivity.

Prior to analysis of the synthesized organic strontium salts, the water solubility of some commercially available strontium salts were determined by the F-AAS method to verify the precision of the measurements and compare the obtained results with reference values for solubility of the salts. The following strontium salts were obtained: Sr— Oxalate (Aldrich 57, 416-3) SrSO₄ (Aldrich 45, 129-0) SrHPO₄ (Aldrich 48, 042-2) and SrCL₂ (Aldrich 43,966-5). The solubilities were investigated as described above, and strontium content in the saturated solutions determined as described here below.

Some of the very soluble strontium salts were further diluted before analysis by F-AAS. The measurements were performed by using a Perkin-Elmer 2100 equipped with a hydrogen lamp for correction of the background signal. Strontium was measured at a slit with of 0.2 nm, the wavelength was 460.8 nm operated at an energy of 58 and a current of 8 mA.

Solutions with very low strontium content (i.e., from the analysis of solubility of strontium carbonate) were analyzed by the inductively coupleD plasma-mass spectrometry (ICP-MS) method. This analysis was performed using a Perkin Elmer Elan 5000 system equipped with a cross-flow nebulizer. The power was set at 1000 W and the Argon-gas flow was 12 L/min and 0.8 L/min of the torch and plasma gas, respectively.

The solubility determined for the commercially available strontium salts were in good agreement with the reference values. For most investigations, the F-AAS method had sufficient sensitivity. Table 4 presents solubilities of strontium chloride, phosphate, carbonate, oxalate and sulphate in water at 22° C. It is apparent that the experimentally determined values are in agreement with the reference values quoted for these salts. The major deviation between reference values and the experiment was obtained for strontium chloride where a lower solubility was obtained and for strontium carbonate where a significantly higher solubility was found. Since the solubility of strontium carbonate is very low, it was necessary to apply ICP-MS to the determination of the content of Sr in the supernatants from these experiments. Furthermore, the solubility of this salt will be dependent on the content of carbon dioxide in the ambient air, which was not controlled in the present experiment, providing one possible explanation for the discrepancies between the determined solubility and the reference value.

TABLE 4
Solubility of commercially available strontium salts in water at room
temperature (22-24°) determined as described in example 3.

| | | Measured | Expected |
|---|---|---|---|
| Salt | Method | g/L | Value 18° C. (g/L) |
| SrCl₂ | F-AAS | 240 | 538 |
| SrHPO₃ | F-AAS | 0.5 | — |
| SrSO₄ | F-AAS | 0.1 | 0.1 |
| SrC₂O₄ | F-AAS | 0.05 | 0.05 |
| SrCO₃ | ICP-MS | 0.00009 | 0.011 |

Expected values refer to values quoted in scientific literature or reference material such as the ‘Beilstein compendium’.

### Temperature and pH Influence on Organic Strontium Salt Solubility

For the majority of the organic strontium salts listed in table 2, temperature changes in the interval from 20-40° C. had only little influence on solubility (table 5). However, for strontium L-glutamate a significant influence of temperature on solubility was observed in the range between 20° C. and 40° C. The solubility of this salt increased more than three-fold in the investigated interval in contrast to most other salts. It is noted, that the solubility under physiological conditions (37° C.), is of relevance for the pharmaceutical use of the substances, and thus the surprising increase in strontium glutamate solubility at higher temperature may have great potential therapeutic implications.

The solubility of the strontium salts in an ammonium carbonate buffered solution of pH 7.5 was generally higher than the solubility determined in pure water (table 5). However, there were some notable exceptions, such as strontium maleate, which had decreased solubility in the buffered solution. Accordingly, it was found most relevant to compare the solubility of the strontium salts by comparing the values obtained in water, as shown in table 5.

### Relative Solubility

The water-solubilities of the organic strontium salts at room temperature and at 40° C., are listed in table 5. The strontium salts of L-aspartate and of lactate had solubilities exceeding 50 g/l hampering exact determination of solubility with the employed experimental procedures.

The results correspond to the observations during the synthesis experiments where the citrate, the fumerate and the tartrate precipitated instantly when synthesized by the production procedures described in examples 1 and 2. This is indicative of a poor solubility of these strontium salts, as apparent by the lower solubility of these salts compared to the other organic strontium salts at both 22° C. and 40° C.

The glutamate salt showed a higher solubility than the other salts, especially at a temperature of 40° C. During the synthesis of this salt, it was necessary to add alcohol to the solution, to initiate crystal growth, indicative of relatively high water solubility. The other studied strontium salts only precipitated after evaporation of the solvent for a few days at room temperature, but addition of alcohol was not required to initiate crystal formation and precipitation.

TABLE 5
Relative solubility in water buffered solutions at pH 7.5
at 40° C. and room temperature (22-24° C.) of the
investigated Strontium-salts, as determined by F-AAS.

| | SOLUBILITY AT ROOM | | SOLUBILITY | |
|---|---|---|---|---|
| STRONTIUM | TEMPERATURE | | AT 40° C. | |
| SALT | (22-24° C.)(Mg/L) | | (mg/L) | |
| Anion | In water | pH 7.5 | In water | pH 7.5 |
| Malonate** | 1474 | 2816 | 1441 | 2127 |
| L-glutamate** | 2111 | 3022 | 7093 | 7195 |
| L-aspartate** | 4200 | | 7900 | |
| Pyruvate* | 2204 | 1946 | 1929 | 1829 |
| α-ketogluterate** | 1316 | 2252 | 3534 | 3809 |
| Fumerate** | 571 | 1215 | 444 | 977 |
| Maleate** | 3002 | 1680 | 2527 | 1457 |
| Tartrate** | 883 | 1831 | 1028 | 1400 |
| Ranelate**** | 760 | 890 | 1450 | 1970 |
| Succinate** | 1137 | 926 | 1116 | 2233 |
| Citrate*** | 107 | 388 | 147 | 430 |

*Mono-carboxylic acid
**Di-carboxylic acid
***Tri-carboxylic acid
****Quattro-carboxylic acid

### Example 4

### Preparation of Strontium Glutamate Hexahydrate by Synthesis at 100° C.

Initially, a suspension of glutamic acid (white colored) is prepared by adding 100 mL of millipore water to 14.703 g (0.1 moles) of solid L-glutamic acid (Sigma Aldrich, C₅H₉NO₄, MW 187.14 g/mole, CAS no. 142-47-2, lot. no. 426560/1, filling code 43003336) in a 250 mL beaker. To this suspension was added 26.571 g (0.1 moles) of solid strontium hydroxide (Sigma Aldrich, Sr(OH)₂*8H₂O, MW 265.71, CAS no. 1311-10-0). Then, a magnetic stirring rod was added and the stirring and heating was started to the point of boiling of the suspension. The final suspension is also white colored and the stirring is sustained by maintaining a medium rotation rate of the stirring apparatus. In order to prevent carbon dioxide from entering the solution, the beaker was covered by a covering glass.

After some minutes of boiling and stirring, the solution clarified and all the solid material dissolved. The boiling was maintained, and additional water was added when required, as to replace the water lost by boiling. After three hours of boiling, the solution was filtered while boiling on a Büchner funnel. Very small amounts of impurities were left in the filter. The filtrate was subsequently allowed to cool to room temperature, which resulted in growth of fine-powdered crystals of strontium glutamate hexahydrate. Precipitation of the final product progressed in the filtrate within an hour. The product was filtered and dried at 110° C. in an oven for ½ hour followed by drying 12 hours in a dessicator over silica orange. Before analysis by x-ray crystallography and by FAAS, the salts were ground to fine powder by a mortar.

The total yield of strontium glutamate hexahydrate was approximately 98% before recrystallisation, and the majority of impurities consisted of reminisces of the reagents and of strontium carbonate. This yield is significantly higher than the yield obtained by synthesis under conventional conditions where only 15% was obtained (please see example B). Thus the high temperature synthesis method as disclosed in this patent provides a significant gain in yield and a reduction in synthesis time, while resulting In a strontium glutamate salt of higher purity. The product was unambiguously identified as strontium glutamate hexahydrate by x-ray crystallography and comparing the data to results of the literature.

Further improvements of the synthesis may include degassing by nitrogen or by argon of the water and of all aqueous solutions, which prevents contact to carbon dioxide that eventually may lead to formation of impurities of strontium carbonate. It follows that a person skilled in the art will easily be able to adapt the procedure to proceed under an inert gas atmosphere.

### Example 5

### Preparation of Strontium Aspartate Trihydrate by Synthesis at 100° C.

Initially, a suspension of aspartic acid (white colored) is prepared by adding 100 mL of millipore water to 13.311 g (0.1 moles) of solid L-aspartic acid (Fluka, C₅H₉NO₄, MW 133.11 g/mole, CAS no. 56-84-8, lot. no. 432866/1, filling code 52603495) in a 250 mL beaker. To this suspension was added 26.571 g (0.1 moles) of solid strontium hydroxide (Sigma Aldrich, Sr(OH)₂*8H₂O, MW 265.71, CAS no. 1311-10-0). Then, a magnetic stirring rod was added and the stirring and heating was started to the point of boiling of the suspension. The final suspension is also white colored and the stirring is sustained by maintaining a medium rotation rate of the stirring apparatus. In order to prevent carbon dioxide from entering the solution, the beaker was covered by a covering glass.

After some minutes of boiling and stirring, the solution clarified and all the solid material dissolved. The boiling was maintained, and additional water was added when required, as to replace the water lost by boiling. After three hours of boiling, the solution was filtered while boiling on a Büchner funnel. Very small amounts of impurities were left in the filter. The filtrate was subsequently allowed to cool to room temperature, which resulted in growth of fine-powdered crystals of strontium aspartate trihydrate. Precipitation of the final product progressed in the filtrate within an hour. The product was filtered and dried at 110° C. in an oven for ½ hour followed by drying 12 hours in a dessicator over silica orange. Before analysis by x-ray crystallography and by FAAS, the salts were ground to fine powder by a mortar.

The total yield of strontium aspartate trihydrate was approximately 98% before recrystallisation, and the majority of impurities consisted of reminisces of the reagents and of strontium carbonate. This yield is significantly higher than the yield obtained by synthesis under conventional conditions where only 14% was obtained (please see example 2). Thus the high temperature synthesis method as disclosed in this patent provides a significant gain in yield and a reduction in synthesis time, while resulting In a strontium aspartate salt of higher purity. The product was unambiguously identified as strontium aspartate trihydrate by x-ray crystallography and comparing the data to results of the Cambridge Crystallographic Database.

Further improvements of the synthesis may include degassing by nitrogen or by argon of the water and of all aqueous solutions, which prevents contact to carbon dioxide that eventually may lead to formation of impurities of strontium carbonate. It follows that a person skilled in the art will easily be able to adapt the procedure to proceed under an inert gas atmosphere.

### Example 6

### Preparation of Strontium Malonate Anhydrous by Synthesis at 100° C.

Initially, a suspension of malonic acid (white colored) is prepared by adding 100 mL of millipore water to 10.406 g (0.1 moles) of solid malonic acid (Fluka, MW 104.06 g/mole, CAS no. 141-82-2, lot. no. 449503/1, filling code 44903076) in a 250 mL beaker. To this suspension was added 26.571 g (0.1 moles) of solid strontium hydroxide (Sigma Aldrich, Sr(OH)2*8H₂O, MW 265.71, CAS no. 1311-10-0). Then, a magnetic stirring rod was added and the stirring and heating was started to the point of boiling of the suspension. The final suspension is also white colored and the stirring was sustained by maintaining a medium rotation rate of the stirring apparatus. In order to prevent carbon dioxide from entering the solution, the beaker was covered by a covering glass.

After some minutes of boiling and stirring, the solution clarified and all the solid material dissolved. The boiling was maintained, and additional water was added when required, as to replace the water lost by boiling. After three hours of boiling, the solution was filtered while boiling on a Büchner funnel. Very small amounts of impurities were left in the filter. The filtrate was subsequently allowed to cool to room temperature, which resulted in growth of fine-powdered crystals of strontium malonate. Precipitation of the final product progressed rapidly during filtration and the majority of the product was found in the filter (unheated). Only in rare instants, the precipitation progressed in the filtrate. The product was filtered and dried at 110° C. in an oven for % 2 hour followed by drying 12 hours in a dessicator over silica orange. Before analysis by x-ray crystallography and by FAAS, the salts were ground to fine powder by a mortar.

The total yield of strontium malonate was approximately 98% before recrystallisation, and the majority of impurities consisted of reminisces of the reagents and of strontium carbonate. The product was unambiguously identified as strontium malonate by x-ray crystallography and comparing the data to results of the Cambridge Crystallographic Database.

Further improvements of the synthesis may include degassing by nitrogen or by argon of the water and of all aqueous solutions, which prevents contact to carbon dioxide that eventually may lead to formation of impurities of strontium carbonate. It follows that a person skilled in the art will easily be able to adapt the procedure to proceed under an inert gas atmosphere.

### Example 7

### Methods of Manufacture of Water Soluble Strontium Salts of Dicarboxylic Acids Using Temperatures above 100° C.

According to methods developed previously and described in examples 2-6, synthesis of strontium salts of dicarboxylic organic acids and especially strontium salts of amino acids can be difficult to produce in larger scale (i.e., >1 kg) due to low yields and difficulties in separating the desired reaction products from contaminants. Strontium salts of carbonate are of special concern, as they will form as impurities when the reaction is occurring in atmospheric air containing normal levels of carbon dioxide. We have described in examples 4-6 that the total yield of the product when strontium salts of dicarboxylic acids are manufactured from the free acid form of the anion, and strontium hydroxide depends on temperature and on time of synthesis. In order for the reaction to reach completion, the mixture of the amino acid proper and strontium hydroxide needs boiling in water for three hours, allowing ample time for strontium in the reaction mixture to react with carbon dioxide in the air. In this example we disclose methods of improving the synthesis further by providing optimized reaction conditions, where temperature is increased above 100° C. in a closed container, and where reaction times are significantly reduced.

The present example provides representative data from the optimization of conditions for synthesis of strontium glutamate in an autoclave system. Strontium glutamate is used as an example, but it the optimizations described in the example is also applicable for the synthesis of other strontium salts, where the exact reaction conditions can be optimized as disclosed in this example. The reaction temperatures must be maintained below the melting point or below the temperature of decomposition of the organic anion moiety of the desired strontium salt. As an example, malonic acid decomposes at 132-134° C., and thus synthesis of strontium malonate must be performed at temperatures below 132° C.

Strontium L-glutamate was used as a model strontium compound in the optimisation experiments. The purity of the product was monitored by comparing to crystallographic data and by measuring the content of strontium. Ideally, the content of strontium is 25.7% in strontium L-glutamate hexahydrate, which is the product formed in these experiments. It follows that other soluble strontium salts maybe prepared by similar methods with high yield and purity.

### Experimental

Preparation of Solutions: a Suspension of Glutamic Acid (White Coloured) is prepared by adding 100 mL of millipore water to 14.703 g (0.1 moles) of solid L-glutamic acid (Sigma Aldrich, C₅H₉NO₄, MW 187.14 g/mole, CAS no. 142-47-2, lot. no. 426560/1, filling code 43003336) in a 250 mL beaker. To this suspension was added 22.257 g, 26.571 g or 31.885 (0.08 moles, 0.1 moles or 0.12 moles) of solid strontium hydroxide (Sigma Aldrich, Sr(OH)₂*8H₂O, MW 265.71, CAS no. 1311-10-0).

### Optimisation Experiments

After preparation of the salts, the nine optimisation experiments were performed according to the settings of table 6.

TABLE 6
Parameters and main results of the optimisation procedure for synthesis of strontium
glutamate. The pressure was monitored but not used in the optimisation process.
The strontium content (% Sr) was measured by FAAS but not used as quality parameter.
The yield (%) was applied as the quality parameter.

| | Actoclave | Time of | | Total | Autoclave | | |
|---|---|---|---|---|---|---|---|
| Experiment | Temperature | Synthesis | Base-acid | Volume | Pressure | | % SR |
| No. | (° C.) | (min.) | Ratio | (ML) | (bar) | Yield % | (AAS) |
| 1 | 125 | 15 | 0.8 | 50 | 1.55 | 94 | 25 |
| 2 | 124 | 30 | 1 | 75 | 1 | 112 | 22 |
| 3 | 124 | 60 | 1.2 | 100 | 1.6 | 121 | 21 |
| 4 | 127 | 15 | 0.8 | 100 | 1.2 | 118 | 22 |
| 5 | 132 | 30 | 1 | 50 | 1.55 | 120 | 25 |
| 6 | 132 | 60 | 1.2 | 75 | 1.6 | 50 | 22 |
| 7 | 134 | 15 | 0.8 | 75 | 1.65 | 108 | 24 |
| 8 | 134 | 30 | 1 | 100 | 1.65 | 76 | 14 |
| 9 | 132 | 60 | 1.2 | 50 | 1.65 | 82 | 24 |

### Procedure

1. The calculated amount of acid was weighed and transferred to a bluecap autoclave bottle and the Millipore water was added. The bottle was closed and shaken, in order to obtain a finely grained suspension.

2. The calculated amount of strontium hydroxide octahydrate was weighed and added to the acid solution of (1) and the bottle was vigorously wortexed until all coarse lumps of material were transformed into fine-grained powder.

3. The bottle was placed in the autoclave and the temperature was set. While in the autoclave no additional stirring was carried out.

4. At t=100° C. the valve of the autoclave was closed and the timing was started.

5. During the autoclaving were monitored the actual temperature and the actual pressure.

6. After the time of autoclaving ended, the steam was let out, as soon as possible, with due respect to safety precautions.

7. At approx. 110° C. the autoclave was opened and the solution was recovered. Again, the bottle was shook, as to obtain a high degree of mixing.

8. The solution was immediately filtered hot on a Büchner funnel after autoclaving, which left only traces of carbonate in the filter. The product precipitated from the solution during cooling to room temperature.

9. After precipitation, the product was filtered and dried in an oven for ½ an hour at 110° C. Then, it was dried in a dessicator over silica-gel orange. Finally, the product was ground to fine powder in a mortar.

10. The product was weighed after grinding and the total yield calculated.

### Preparation of Strontium Malonate According to the Invention

In order to confirm the applicability of the disclosed high temperature synthesis method for other strontium salts than strontium L-glutamate, strontium malonate was prepared. Basically the reaction conditions found for preparation of strontium L-glutamate was employed. A suspension of malonic acid (white coloured) is prepared by adding 100 mL of millipore water to 10.41 g (0.1 moles) of solid malonic acid (FLUKA 63290, MW 104.1) in a 250 mL beaker. To this suspension was added 22.257 g, 26.571 g or 31.885 (0.08 moles, 0.1 moles or 0.12 moles) of solid strontium hydroxide (Sigma Aldrich, Sr(OH)₂*8H₂O, MW 265.71, CAS no. 1311-10-0). The reaction procedure described above was follower, and the temperature was maintained below 130° C. to avoid decomposition of malonic acid, while the reaction time was maintained at 15 min.

### Content of Strontium (% Sr):

A sample of 0.2 g was dissolved in 100 mL 0.1 M HNO₃ prepared in Millipore water. This solution was further diluted by a factor of 500 by a solution of 1% KCl, and the content of strontium was determined by FAAS. The measurements were performed by using a Perkin-Elmer 2100 equipped with a hydrogen lamp for correction of the background signal. Strontium was measured at a slit with of 0.2 nm, the wavelength was 460.8 nm operated at an energy of 58 and a current of 8 mA.

### X-Ray Crystallography

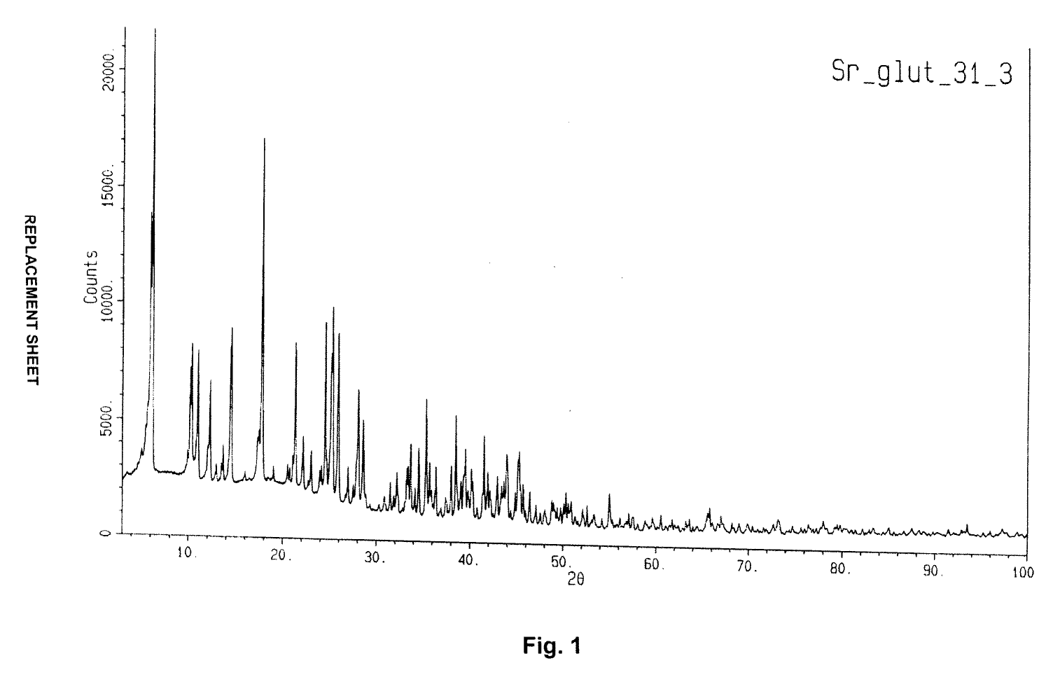

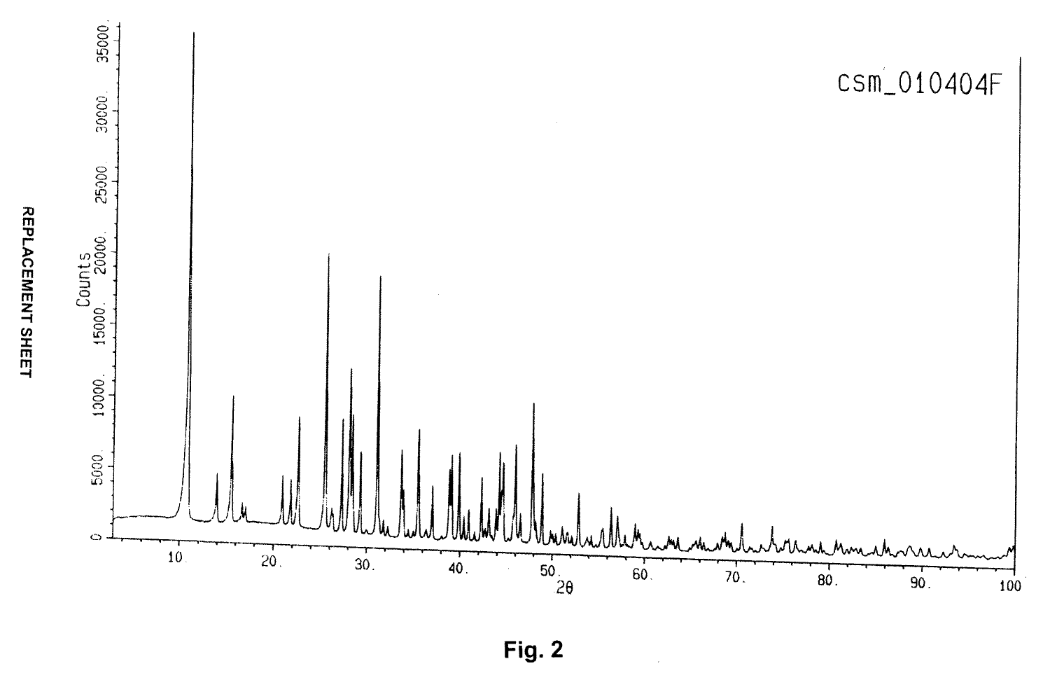

A second check of purity was performed by powder x-ray crystallography using a Huber G670 diffractometer. A characteristic diffractogram of the strontium glutamate is shown in FIG. 1. An X-ray diffractogram of strontium malonate obtained by the high temperature synthesis method disclosed in the present example is shown in FIG. 2. The double peak on the low angle side of the peak of maximum intensity is an artifact of the instrument.

### Results and Discussion

In table 4, it is observed that some of the synthesis conditions resulted in relatively low yield and in strontium glutamate of low purity as apparent from the molar % of strontium in the reaction product. The product of experiment no. 8 was produced in relatively low yield, and it did not contain the expected 25.7% of strontium, which was also confirmed by the x-ray analysis. Despite this outlier, in general, the outcome of the optimisation experiments is close to the expected products. Incomplete reaction provides a product of too low content of strontium while formation of strontium carbonate during the synthesis gives a too high value of the strontium content. Conditions employed in experiments 1 and 5 gave the strontium content in best agreement with the expected value. Of notice, it is also apparent although the product of experiment no. 6 was produced in low yield, it contained an amount of strontium that corresponded to the expected value.

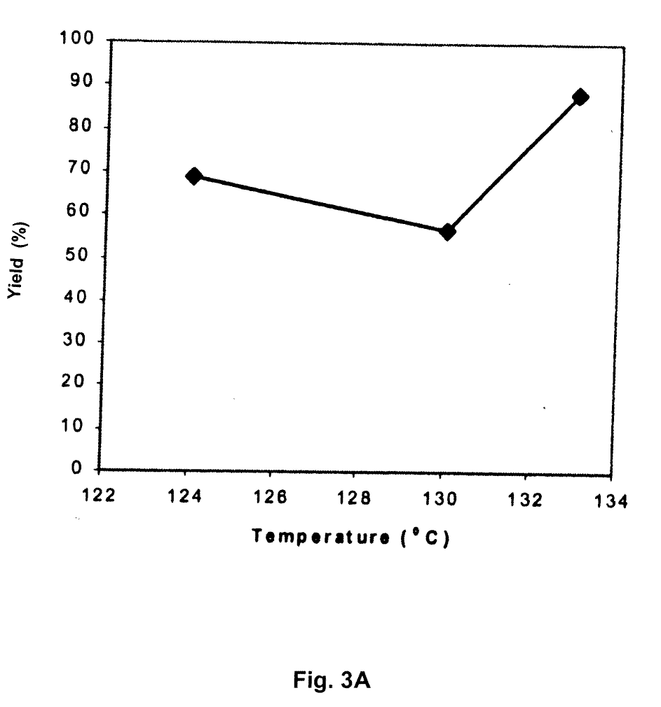

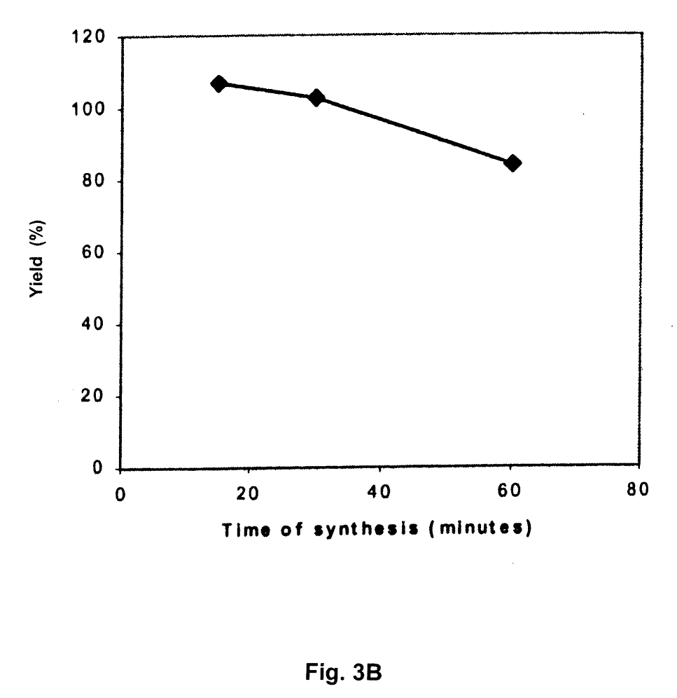

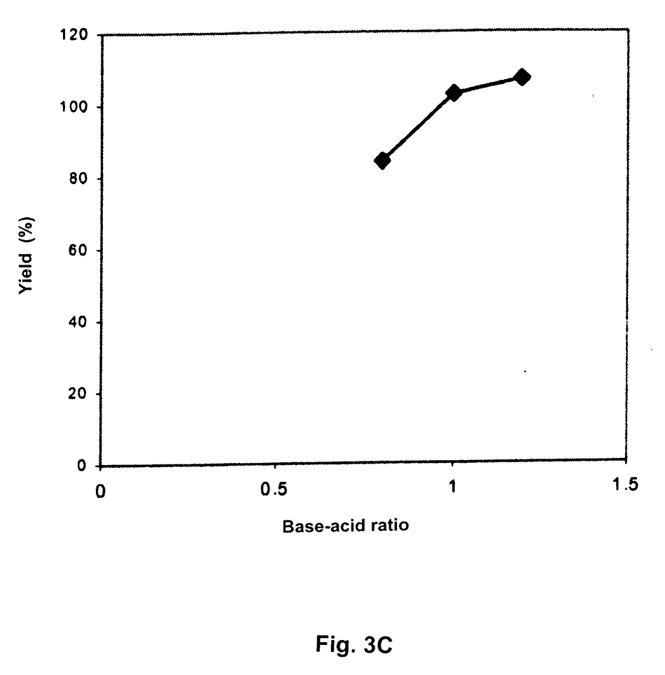

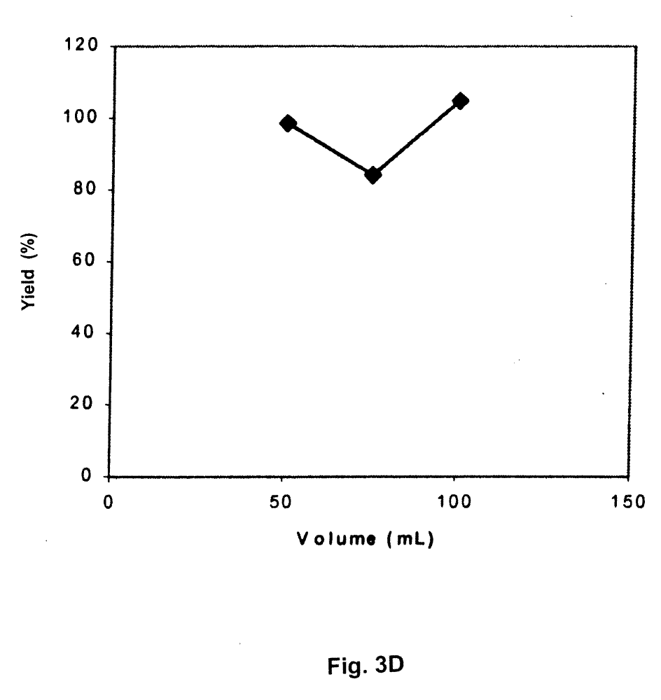

By studying the influence of the individual parameters on the total yield (table 6 and FIG. 3), it becomes clear that temperature, time of autoclaving and base-acid ratio are important for the synthesis while total volume is less important. A yield higher than 100%, which is observed in experimental conditions 2, 3, 4, 5 and 7 originates from incomplete drying, but this effect is almost eliminated when the average values are considered, as in FIG. 3. Thus, the maximum yield was obtained by using a high temperature (133° C.), a short time of autoclaving (15 min.) and a surplus of strontium hydroxide. Accordingly, temperature is more important than time but it compares in importance to the base-to-acid ratio. However, great care must exerted as to not exceed the temperature of decomposition in the synthesis of other strontium salts, which for, e.g., the malonate is 132-134° C. A 10th experiment of control of optimisation was performed, as to confirm the maximum yield of the optimisation experiments.

Furthermore, an additional experiment was performed to validate the applicability of the high temperature synthesis method for the preparation of other organic strontium salts than strontium L-glutamate. Strontium malonate was chosen, as this salt may be considered especially difficult to prepare under the high temperature conditions due to the low dissociation temperature of the malonic acid anion. However, as shown in FIG. 2, crystalline pure and well-defined strontium malonate could easily be obtained.

Further improvements of the synthesis include introduction of inert atmospheres to the synthesis environment, as well as degassing of all solutions by either nitrogen gas or by argon gas, as to reduce the formation of strontium carbonate.

### Conclusion

The optimisation experiments show that it is possible to synthesize strontium glutamate in high yields by elevating the temperature to values above 100° C., and by using a short time (15 min.) in the autoclave. Also, a 20% surplus of strontium-hydroxide also improves the total yield without compromising the purity of the synthesized strontium salt. A slightly more vigorous drying than silica-gel orange should be applied to the drying procedure in order to obtain completely dried product. Examples of more potent drying agents are concentrated sulphuric acid or calcium oxide, but also conventional lyophilization or other mechanic treatments may be applicable for this procedure.

### Example 8

### Pharmacokinetic Properties of Organic Strontium Salts with Low Solubility

The aim of this experiment was to assess the bioavailability of an organic strontium salt with low solubility (strontium citrate) compared with strontium chloride and strontium ranelate. The bioavailability was assessed by determination of serum strontium concentration at regular intervals over a 24 hour period and calculating AUC.

The experiment was performed with female SPF Wistar rats of the strain HanTac:WH (GALAS) from Taconic M&B A/S, Ejby, DK-4623 Lille Skensved, Denmark. At the start of the acclimatisation period, the rats were approximately 9 weeks old with a weight of approximately 200-250 g. The animals were housed in a room provided with filtered air at a temperature of 21° C.±3° C. and relative humidity of 55%±15% and a ventilation system providing 10 air changes per hour. The room was illuminated to give a cycle of 12 hours light and 12 hours darkness. The rats were fed a complete pelleted rodent diet “Altromin 1314” (Chr. Petersen A/S, DK-4100 Ringsted, Denmark). The rats had free access to bottles with domestic quality drinking water acidified with hydrochloric acid to pH 2.5 in order to prevent microbial growth.

The rats were randomly allocated randomly in four groups of 9 animals treated as indicated in the table below. The groups, dose levels, animal numbers were as listed in the table 7 below:

TABLE 7
The 4 treatment groups of the pharmacokinetic experiment. The
doses administered to each group is listed in the fist column,
and salt, MW and Sr content in the middle columns.

| Dose¹ | | Strontium | | | Dose Equivalent¹ | Animal |
|---|---|---|---|---|---|---|
| (mg/kg) | Group | Salt | MW | % Sr | (Amounts in mg) | No's |
| Vehicle | Control | Vehicle | — | — | — | 1-9 |
| | | (0.5% | | | | |
| | | CMC) | | | | |
| 500 | B | Sr- | 639.6 | 27.4 | 500 = 137 mg | 10-18 |
| | | ranelate | | | Sr⁺⁺ | |
| | | (*7H₂O) | | | | |
| 416 | C | SrCl₂ | 266.6 | 32.9 | 137 mg Sr⁺⁺ = | 19-27 |
| | | (*6H₂O) | | | 416 | |
| 390 | D | Sr-citrate | 749.1 | 35.1 | 137 mg Sr⁺⁺ = | 28-36 |
| | | (*6H₂O) | | | 390 | |

¹Doses are adjusted to provide equimolar strontium dose as 500 mg/kg Strontiumranelate (heptahydrate)(group B).

The test article was given once by oral gavage according to the most recent body weight data. The control group was dosed with the vehicle alone (0.5% carboxy methyl cellulose, CMC). The vehicle was prepared with de-ionized water for all treatment groups including controls. The test substances (strontium salts) were solubilized/suspended in a volume corresponding to 5 ml/kg body weight. In order to keep the compounds in suspension, the formulations were kept on a magnetic stirrer before and during dosing.

### Blood Samples for Determination of Strontium Absorption and Bioavailability

On the day of treatment (Day 1), blood samples were taken from all animals. Blood samples were collected from 3 animals per group at the following time points: Pre-treatment, and 30 min, 1, 1.5, 2, 4, 8 and 24 hours post-treatment, so that three animals from each group had samples taken at time 0, 1.5 and 6 hours, 3 other rats at time 0.5, 2, 8 hours and the remaining three animals in the group had samples taken at 1, 4 and 24 hours.

Approximately 0.5-0.6 ml blood was obtained at each time point from the orbital venous plexus into plain tubes for serum. The blood was kept at room temperature for 30 to 60 minutes and until centrifugation (10 min, 1270 G, +20° C.). The serum was transferred to Nunc cryotubes (Nunc, Denmark) and frozen at −18° C. for subsequent analysis of strontium content by graphite-furnace atomic-absorption spectrometry (GF-AAS).

### Graphite-Furnace Atomic-Absorption Spectrometry (GF-AAS)

Concentrated HCI was added to the serum samples to a final concentration of 0.2% HCl and the samples were then subjected to analysis using a Perkin-Elmer 2100 equipped with a hydrogen lamp for correction of the background signal. Strontium was measured at a slit with of 0.2 nm, the wavelength was 460.8 nm operated at an energy of 58 and a current of 8 mA.

### Results of the Pharmacokinetic Study of Strontium Salt Absorption

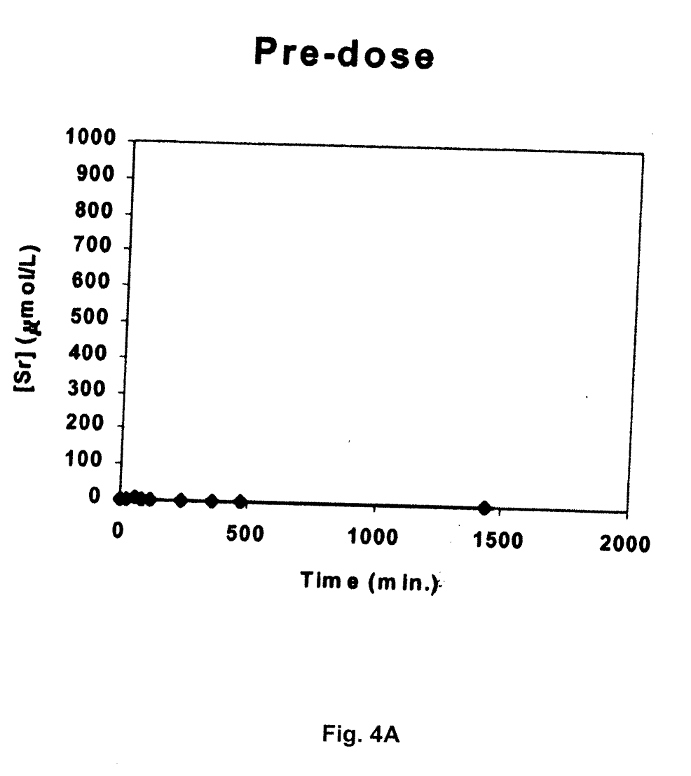

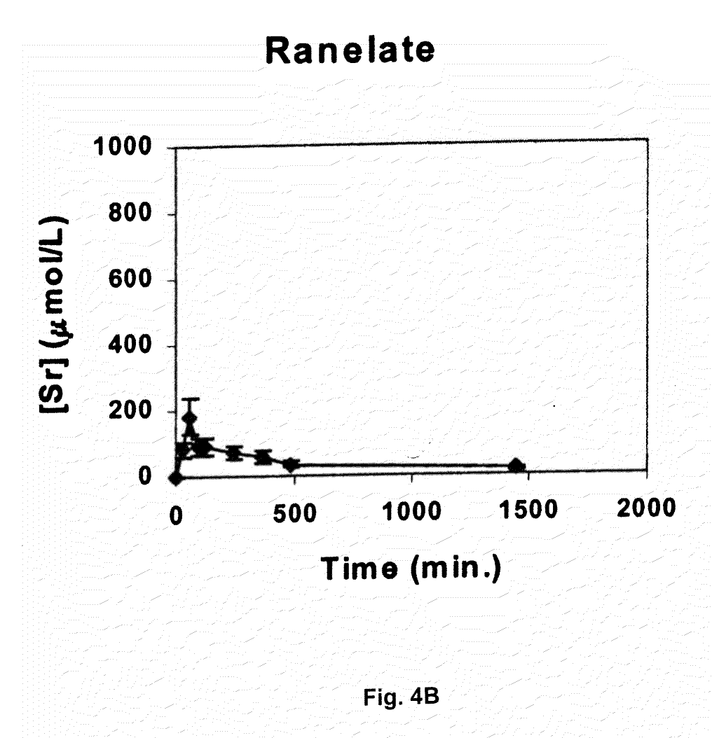

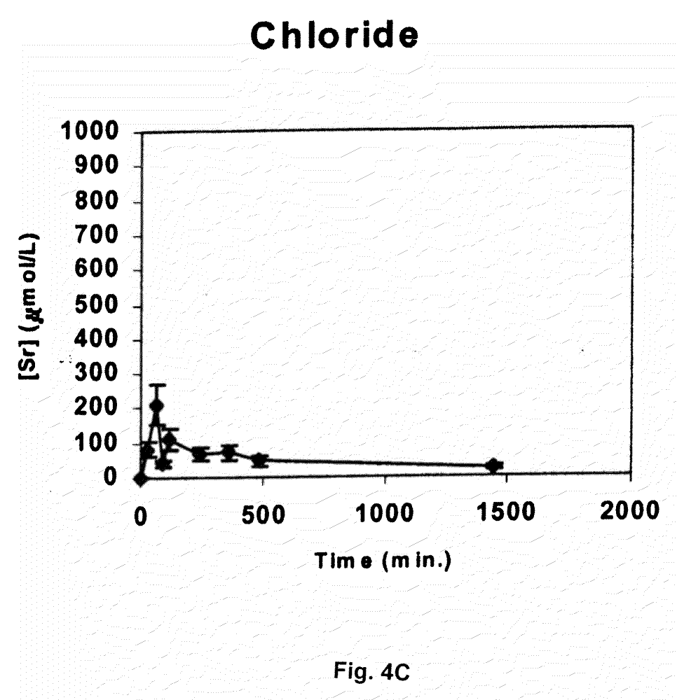

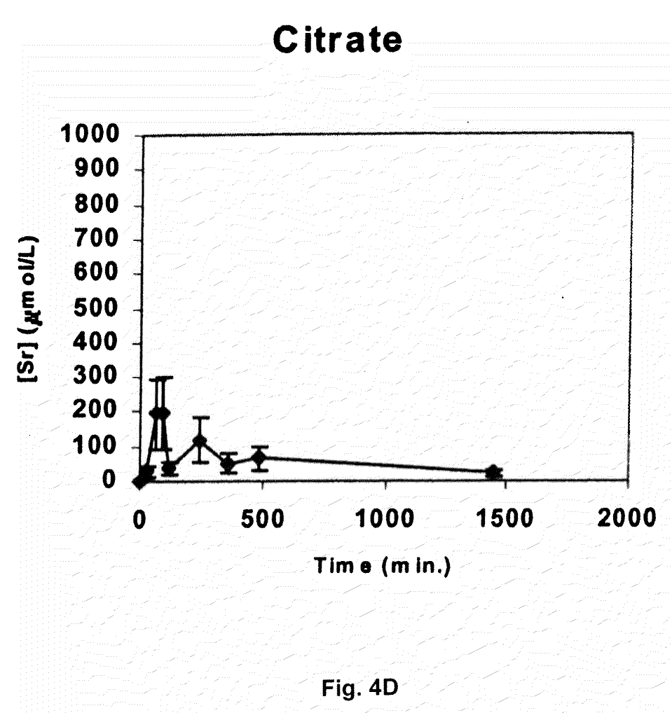

In FIG. 4, the serum concentration measured in the three groups treated with strontium salts are plotted as a function of the time after administration of the compounds. It is apparent that administration of the strontium salts results in a rapid and highly significant increase in serum strontium concentrations. When comparing the pharmaco-kinetic properties of different salts, it is apparent that both the highly soluble strontium chloride as well as the relatively poorly soluble strontium ranelate (see example 3), is rapidly absorbed, reaching a maximum serum concentration after approximately 2 hours. In contrast strontium citrate with the lowest solubility reaches the maximal serum concentration with a slower kinetic rate and, with maximal concentration reached after approximately 6-8 hours. Furthermore, the serum strontium concentration in the time interval from 0-8 hours after the administration of strontium citrate appears more stable.

When AUC calculations were performed the general course of the curves, as evidenced by average values in FIG. 4, was best described by modelling the response/pharmacokinetic curves in a specially developed mathematical model. In the initial step, it assumes that the strontium is not metabolised but simply transferred from the stomach/upper digestive tract of the rat into epithelial cells by an active transport mechanism. Also without metabolism, the strontium ion is then transferred from the stomach/upper digestive tract where it is simultaneously released to the blood vessels. Only during the circulation of strontium through the veins, the strontium is dispersed and metabolised by the body tissue. This credible but simplified description thus includes a two-step mechanism of absorption of ionic strontium after oral administrations of strontium ions, probably corresponding to two uptake mechanisms, an active rapidly activated mechanism, and a passive transport mechanism active throughout the length of the digestive tract. After the strontium dose was administered to the rats, a characteristic time of uptake was found as t=12 min. The maximum content of strontium in the serum was observed after approx. 30 min. The characteristic time value of 12 min. is interpreted as the duration of strontium ions being taken up by the active transport mechanism from the intestinal lumen and secreted into circulation. The time of strontium transfer between the stomach and the blood vessels is initiated almost instantly, while the time of transfer between the guts and the blood vessels proceeds at a later stage that depends on the type of salt investigated. For all salts, however, the strontium content levels out after approx. 1750 min. (29 hours) and approaches the natural level corresponding to the pre-dose level.

The model calculations were applied to the determination of the areas under the curve that are shown in table 7. The standard deviations of the AUC values correspond to the general uncertainty on the measurements of FIG. 4, and their magnitude does not allow for a significant discrimination between the salts.

TABLE 7
Determination of the area under the curve
according (AUC) to the model calculations.

| ANION OF Sr- | AUC | STDDEV |
|---|---|---|
| SALT | mg/L · min | mg/L · min |
| Chloride | 7300 | 2000 |
| Citrate | 8900 | 4700 |
| Vehicle | 168 | 67 |
| Ranelate | 5800 | 1700 |

These effects of delayed uptake of strontium observed with strontium citrate may enhance the pharmacologic properties of strontium. Strontium citrate resulted in the highest level of bioavailability as assessed from the AUC curve (table 7), although the differences to the other treatment groups did not reach statistical significance. The delayed attainment of Cₘₐₓ may be an advantage for the use of the strontium compound in the treatment of diseases and conditions affecting bone metabolism. In these cases it is often an advantage to administer the compound in the evening before bedtime, as this would allow the compound to act at night, when resorption of bone is occurring at the highest rate. Furthermore, the administration before bedtime minimizes the potential interference from calcium in the normal diet, as the pharmaceutical preparation of the strontium salt would be taken after the last meal. This is in contrast to administration during the day, where the calcium content of normal meals would have the potential to interfere and reduce the uptake of strontium. The gradual increase in serum strontium concentration over 4-8 hours after administration of the compound would comply well with evening administration of the compound and appears well suited to maximize the therapeutic effect of the strontium compound on bone metabolism.

What is claimed is:

1. A controlled release pharmaceutical composition for oral use comprising a strontium salt.

2. The pharmaceutical composition according to claim 1, wherein the water-solubility of the strontium salt is at the most about 200 g/l at room temperature (20-25° C.).

3. The pharmaceutical composition according to claim 1, wherein the water solubility of the strontium salt is at least 0.1 g/l at room temperature (20-25° C.).

4. The pharmaceutical composition according to claim 1, wherein the water solubility of the strontium salt is at least 1 g/l at room temperature of (20-25° C.).

5. The pharmaceutical composition according to claim 1 for administration once daily at bed-time.

6. The pharmaceutical composition according to claim 1 comprising at least 0.01 g of strontium, wherein the strontium is calculated as strontium ion.

7. The pharmaceutical composition according to claim 1, comprising at least about 0.5 g of strontium, wherein the strontium is calculated as ionic strontium ion.

8. The pharmaceutical composition according to claim 1, wherein the strontium salt is released from the composition in such a manner that the amplitude (difference between peak and nadir) of the plasma concentration relative to the peak level is less than about 40%.

9. The pharmaceutical composition according to claim 1, wherein the composition when tested in an in vitro dissolution test releases strontium ion from the strontium salt containing composition in the following manner:
within the first 30 minutes of the test at the most about 10% w/w of the strontium ion is released;
within the first 4 hours of the test at the most about 70% w/w of the strontium ion is released; and
within the first 14 hours of the test about 70% w/w or more of the strontium ion is released.

10. The pharmaceutical composition according to claim 1, wherein the strontium salt is contained in a matrix that governs the release.

11. The pharmaceutical composition according to claim 1, wherein the composition is coated with a controlled release coating governing the release of the strontium salt.

12. The pharmaceutical composition according to claim 1, wherein the strontium salt comprises strontium salts of an organic or an inorganic acid.

13. The pharmaceutical composition according to claim 12, wherein the inorganic acid comprises hydrofluoric acid, hydrochloric acid, hydrobromic acid, hydroiodic acid, nitric acid, nitrous acid, phosphoric acid, phosphinic acid, phosphonic acid, sulfonic acid, sulfuric acid, sulfurous acid, disulfuric acid or boric acid.

14. The pharmaceutical composition according to claim 12, wherein the organic acid comprises acetic acid, C₂H₅COOH, C₃H₇COOH, C₄H₉COOH, (COOH)₂, CH₂(COOH)₂, C₂H₄(COOH)₂, C₃H₆(COOH)₂, C₄H₈(COOH)₂, C₅H, (COOH)₂, fumaric acid, maleic acid, malonic acid, lactic acid, citric acid, tartaric acid, oxalic acid, ascorbic acid, benzoic acid, salicylic acid, phthalic acid, carbonic acid, formic acid, acid, L- and D-glutamic acid, L- and D-aspartic acid, glucosamine sulphate, L-threonate, trifluoroacetic acid or ranelic acid.

15. The pharmaceutical composition according to claim 12, wherein the acid is a non-chelator of strontium.

16. The pharmaceutical composition according to claim 12, wherein the salt is in hydrate, anhydrous, solvate, polymorphous, amorphous, crystalline, microcrystalline or polymeric form.

17. The pharmaceutical composition according to claim 11, wherein the salt comprises strontium citrate, strontium succinate, strontium fumarate, strontium ascorbate, strontium tartrate, strontium glutarate, strontium malonate, strontium methanesulfonate, strontium benzenesulfonate, strontium glucosamine sulphate, strontium L-threonate, or mixtures thereof.

18. The pharmaceutical composition according to claim 12, wherein the anion of the strontium salts acid is derived from a monoprotic, a diprotic or a triprotic acid.

19. A method for treatment and/or prophylaxis of a cartilage and/or bone disease and/or conditions resulting in a dysregulation of cartilage and/or bone metabolism in a mammal, the method comprising administering a single daily dose of a controlled release pharmaceutical composition comprising a strontium salt, wherein the amount of strontium salt is adjusted so that the pharmaceutical composition is suitable for administration once daily.

20. A method for treatment and/or prophylaxis of a cartilage and/or bone disease and/or conditions resulting in a dysregulation of cartilage and/or bone metabolism in a male mammal, the method comprising administering a controlled release pharmaceutical composition comprising a strontium salt, wherein the amount of strontium salt is adjusted so that the pharmaceutical composition is suitable for administration once daily.

21. A method for preventing in a subject a cartilage and/or bone disease and/or conditions resulting in a dysregulation of cartilage and/or bone metabolism in a mammal, the method comprising administering a controlled release pharmaceutical composition comprising a strontium salt, wherein the amount of strontium salt is adjusted so that the pharmaceutical composition is suitable for administration once daily.

22. The method according to claim 21, wherein the subject is a female having a bone mineral density, BMD, of more than 1 SD below the young adult female mean.

23. The method according to claim 21, wherein the subject is a female having a BMD below the adult female mean for women of the same age.

24. The method according to claim 21, wherein the subject is a male having a BMD of more than 1 SD below the young adult male mean.

25. The method according to claim 21, wherein the subject is a male having a BMD below the adult male mean for men of the same age.

26. The method according to claim 21, wherein the subject is a female having a level of a specific biomarker of bone resorption, of more than 1 SD above the young adult female mean.

27. The method according to claim 21, wherein the subject is a female having a level of a specific biomarker of bone resorption above the adult female mean for women of the same age.

28. The method according to claim 21, wherein the subject is a male having a level of a specific biomarker of bone resorption, of more than 1 SD above the young adult male mean.

29. The method according to claim 21, wherein the subject is a male having a level of a specific biomarker of bone resorption above the adult mean for men of the same age.

30. The method according to claim 21, wherein the subject is a 20 year or older female.

31. The method according to claim 21, wherein the subject is a female that is about the same age as her age of onset of menopause.

32. The method according to claim 21, wherein the subject is a female who is about 6 months or more beyond the onset of menopause.

33. The method according to claim 21, wherein the subject is a 20 year or older male.

34. The method according to claim 21, wherein the daily dose of strontium administered is at least 0.01 g of strontium.

35. The method according to claim 21, wherein the method further comprises administering an amount of calcium to a subject in need thereof.

36. The method according to claim 35 wherein the daily dose of calcium is at least about 0.01 g.

37. The method according to claim 36, wherein calcium is administered at least 0.5 h after the administration of the strontium component.

38. The method according to claim 35, wherein calcium is administered at least 0.5 h before the administration of the strontium component.

39. The method according to claim 21, wherein the method further comprises administering an amount of vitamin D to a subject in need thereof.

40. The method according to claim 39, wherein the vitamin is vitamin D₃ and the daily dose is at least about 1 μg.

41. The method according to claim 40, wherein the daily dose of vitamin D₃ is from about 5 μg to about 30 μg.

42. The method according to claim 39, wherein vitamin D is vitamin D₂, and the daily dose of vitamin D₂ is at least 1 μg.

43. The method according to claim 42, wherein the daily dose of vitamin D₂ is from about 5 μg to about 125 μg.

44. The method according to claim 39, wherein the strontium and the vitamin D components are administered simultaneously.

45. A method for treating and/or preventing secondary osteoporosis in a subject, the method comprising administering an effective amount of a strontium salt to the subject.

46. The method according to claim 45, wherein the secondary osteoporosis is induced by endocrine diseases, metabolic causes, nutritional conditions, drug substances and/or disorders of the collagen metabolism.

47. A method for preventing drug induced secondary osteoporosis in a subject, the method comprising administering to the subject a prophylactic amount of a strontium salt before, during or after treatment of the subject with the drug substance that induces secondary osteoporosis.

48. The method according to claim 47, wherein the administration takes place substantially simultaneously with administration of the drug substance that induces osteoporosis.

49. The method according to claim 48, wherein the strontium salt and the drug substance that induces osteoporosis are contained in the same pharmaceutical composition.

50. A pharmaceutical composition comprising a strontium salt and a drug substance that induces osteoporosis together with a pharmaceutically acceptable excipient.

51. The method according to claim 19, wherein the cartilage and/or bone disease and/or conditions resulting in a dysregulation of cartilage and/or bone metabolism in a mammal, comprises osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues or for weight gain.

52. The method according to claim 20, wherein the cartilage and/or bone disease and/or conditions resulting in a dysregulation of cartilage and/or bone metabolism in a mammal, comprises osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, for the improvement of implant stability and for the maintenance or increase of energy level, for building up or strengthening muscle tissues or for weight gain.

53. The method according to claim 21, wherein the cartilage and/or bone disease and/or conditions resulting in a dysregulation of cartilage and/or bone metabolism, comprises osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, for the improvement of fracture healing after traumatic or atraumatic fracture, and for the maintenance or increase of energy level, for building up or strengthening muscle tissues or for weight gain.

54. The pharmaceutical composition of claim 8, wherein the repeated administration comprises repeated administration of the composition to the subject once daily for at least seven days.

55. The method of claim 19, wherein the strontium salt comprises at least 0.5 g of strontium.

56. The method of claim 19, wherein the pharmaceutical composition is administered orally.

57. The method of claim 20, wherein the pharmaceutical composition is administered in an amount and frequency that gives a daily dose of from about 0.25 g to about 1.5 g strontium ion.

58. The method of claim 20, wherein the pharmaceutical composition is administered orally.

59. The method of claim 21, wherein the pharmaceutical composition is administered in an amount and frequency that gives a daily dose of from about 0.25 g to about 1.5 g strontium ion.

60. The method of claim 21, wherein the pharmaceutical composition is administered orally.
